(19) United States
(12) Patent Application Publication
(10) Pub. No.: US 2025/0001110 A1
(43) Pub. Date: Jan. 2, 2025

(54) SYSTEMS AND METHODS FOR OPTIMIZING PARAMETERS OF A RESPIRATORY THERAPY SYSTEM

(71) Applicant: ResMed Pty Ltd, Bella Vista (AU)

(72) Inventors: Nathan Zersee Liu, Bella Vista (AU); Chinh Duc Nguyen, Bella Vista (AU); Vinh Phuc Tran, Bella Vista (AU); Hussein Emami, San Diego, CA (US)

(21) Appl. No.: 18/883,655

(22) Filed: Sep. 12, 2024

Related U.S. Application Data
(63) Continuation-in-part of application No. PCT/AU2023/050179, filed on Mar. 15, 2023.
(60) Provisional application No. 63/651,846, filed on May 24, 2024.
(60) Provisional application No. 63/320,085, filed on Mar. 15, 2022.

(51) Int. Cl. A61M 16/00

(52) U.S. Cl. CPC A61M 16/026; A61M 16/0003

(57) ABSTRACT

A method of optimizing a plurality of parameters of a respiratory therapy system comprises receiving user data associated with a user of the respiratory therapy system. The method further comprises determining an initial value of each of the plurality of parameters is based at least in part on the user data. Each of the plurality of parameters is associated with a comfort level of the user. The method further comprises receiving usage data associated with use of the respiratory therapy system during a first period of time in which each of the plurality of parameters has its initial value. The method further comprises generating a recommended value of each of the plurality of parameters for use of the respiratory therapy system during a second period of time after the first period of time, based at least in part on the user data and the usage data.

### CROSS REFERENCE TO RELATED APPLICATIONS

This application claims the benefit of, and priority to, U.S. Provisional Patent Application No. 63/651,846, filed on May 24, 2024, and is a continuation-in-part of International Application No. PCT/AU2023/050179, filed on Mar. 15, 2023, which claims the benefit of, and priority to, U.S. Provisional Patent Application No. 63/320,085, filed on Mar. 15, 2022, each of which is hereby incorporated by reference herein in its entirety.

### TECHNICAL FIELD

The present disclosure relates generally to systems and methods for optimizing parameters of a respiratory therapy system, and more particularly, to systems and methods for optimizing parameters of a respiratory therapy system based at least in part on user-associated data and usage data.

### BACKGROUND

Many individuals suffer from sleep-related and/or respiratory disorders such as, for example, Sleep-Disordered Breathing (SDB), which can include Obstructive Sleep Apnea (OSA), Central Sleep Apnea (CSA), other types of apneas such as mixed apneas and hypopneas, and Respiratory Effort Related Arousal (RERA). These individuals may also suffer from other health conditions (which may be referred to as comorbidities), such as insomnia (characterized by, for example, difficult in initiating sleep, frequent or prolonged awakenings after initially falling asleep, and/or an early awakening with an inability to return to sleep), Periodic Limb Movement Disorder (PLMD), Restless Leg Syndrome (RLS), Cheyne-Stokes Respiration (CSR), respiratory insufficiency, Obesity Hyperventilation Syndrome (OHS), Chronic Obstructive Pulmonary Disease (COPD), Neuromuscular Disease (NMD), rapid eye movement (REM) behavior disorder (also referred to as RBD), dream enactment behavior (DEB), hypertension, diabetes, stroke, and chest wall disorders. These individuals are often treated using a respiratory therapy system (e.g., a continuous positive airway pressure (CPAP) system), which delivers pressurized air to aid in preventing the individual's airway from narrowing or collapsing during sleep. The respiratory therapy system can include a conduit that delivers the pressurized air from a respiratory therapy device having a flow generator (e.g., a motor), to a user interface coupled to the individual's face. In certain situations, the user may have less than desirable compliance with the user's prescribed use of the respiratory therapy system. The present disclosure is directed to solving this and other problems.

### SUMMARY

According to some implementations of the present disclosure, a method of optimizing a plurality of parameters of a respiratory therapy system comprises receiving data associated with a user of the respiratory therapy system. The method further comprises determining an initial value of each of the plurality of parameters. Each of the plurality of parameters is associated with a comfort level of the user. The determination of the initial values is based at least in part on the user data. The method further comprises receiving usage data associated with use of the respiratory therapy system during a first period of time. During use of the respiratory therapy system in the first period of time, each of the plurality of parameters has its initial value. The method further comprises generating a recommended value of each of the plurality of parameters for use of the respiratory therapy system during a second period of time after the first period of time. The generation of the recommended values is based at least in part on the user data and the usage data.

According to some implementations of the present disclosure, a method of optimizing a combination of two or more parameters of a respiratory therapy system comprises receiving data associated with a user of the respiratory therapy system. The method further comprises determining an initial value of each parameter of the combination of two or more parameters. Each parameter of the combination of two or more parameters is associated with a comfort level of the user. The determination of the initial values is based at least in part on the user data. The method further comprises receiving usage data associated with use of the respiratory therapy system during a first period of time. During use of the respiratory therapy system in the first period of time, each parameter of the combination of two or more parameters has its initial value. The method further comprises generating a recommended value of each parameter of the combination of two or more parameters for use of the respiratory therapy system during a second period of time after the first period of time. The generation of the recommended values is based at least in part on the user data and the usage data.

According to some implementations of the present disclosure, a system comprises a control system and a memory. The control system includes one or more processors. The memory stores machine-readable instructions. The control system is coupled to the memory device, and is configured to execute the machine-readable instructions to implement a method of optimizing a plurality of parameters of a respiratory therapy system. The method includes receiving data associated with a user of the respiratory therapy system. The method further includes determining an initial value of each of the plurality of parameters. The determination of the initial values is based at least in part on the user data. The method further includes receiving usage data associated with use of the respiratory therapy system during a first period of time. During use of the respiratory therapy system in the first period of time, each of the plurality of parameters has its initial value. The method further comprises generating a recommended value of each of the plurality of parameters for use of the respiratory therapy system during a second period of time after the first period of time. The generation of the recommended values is based at least in part on the user data and the usage data.

According to some implementations of the present disclosure, a system comprises a control system and a memory. The control system includes one or more processors. The memory stores machine-readable instructions. The control system is coupled to the memory device, and is configured to execute the machine-readable instructions to implement a method of optimizing a combination of two or more parameters of a respiratory therapy system. The method includes receiving data associated with a user of the respiratory therapy system. The method further includes determining an initial value of each parameter of the combination of two or more parameters. The determination of the initial values is based at least in part on the user data. The method further includes receiving usage data associated with use of the respiratory therapy system during a first period of time. During use of the respiratory therapy system in the first period of time, each parameter of the combination of two or more parameters has its initial value. The method further comprises generating a recommended value of each parameter of the combination of two or more parameters for use of the respiratory therapy system during a second period of time after the first period of time. The generation of the recommended values is based at least in part on the user data and the usage data.

According to some implementations of the present disclosure, a system comprises a respiratory therapy system, a memory device, and a control system. The respiratory therapy system is configured to supply pressurized air to an individual. The memory device stores thereon machine-readable instructions. The control system includes one or more processors that are configured to execute the machine-readable instructions to implement a method. The method includes receiving data associated with a user of the respiratory therapy system. The method further includes determining an initial value of each of a plurality of parameters of the respiratory therapy system. Each of the plurality of parameters is associated with a comfort level of the user. The determination of the initial values is based at least in part on the user data. The method further includes receiving usage data associated with use of the respiratory therapy system during a first period of time. During use of the respiratory therapy system in the first period of time, each of the plurality of parameters has its initial value. The method further includes generating a recommended value of each of the plurality of parameters for use of the respiratory therapy system during a second period of time after the first period of time. The generation of the recommended values is based at least in part on the user data and the usage data.

According to some implementations of the present disclosure, a system comprises a respiratory therapy system, a memory device, and a control system. The respiratory therapy system is configured to supply pressurized air to an individual. The memory device stores thereon machine-readable instructions. The control system includes one or more processors that are configured to execute the machine-readable instructions to implement a method. The method includes receiving data associated with a user of the respiratory therapy system. The method further includes determining an initial value of each parameter of a combination of two or more parameters of the respiratory therapy system. Each parameter of the combination of two or more parameters is associated with a comfort level of the user. The determination of the initial values is based at least in part on the user data. The method further includes receiving usage data associated with use of the respiratory therapy system during a first period of time. During use of the respiratory therapy system in the first period of time, each parameter of the combination of two or more parameters has its initial value. The method further includes generating a recommended value of each parameter of the combination of two or more parameters for use of the respiratory therapy system during a second period of time after the first period of time. The generation of the recommended values is based at least in part on the user data and the usage data.

The above summary is not intended to represent each embodiment or every aspect of the present invention. Additional features and benefits of the present invention are apparent from the detailed description and figures set forth below.

### BRIEF DESCRIPTION OF THE DRAWINGS

FIG. 1 is a functional block diagram of a system for detecting rainout in a respiratory therapy system, according to some implementations of the present disclosure;

FIG. 2 is a perspective view of the system of FIG. 1, a user of the system, and a bed partner of the user, according to some implementations of the present disclosure;

FIG. 3 illustrates an exemplary timeline for a sleep session, according to some implementations of the present disclosure;

FIG. 4 illustrates an exemplary hypnogram associated with the sleep session of FIG. 3, according to some implementations of the present disclosure;

FIG. 5 is a process flow diagram of a method for optimizing sleep for a user of a respiratory therapy system, according to some implementations of the present disclosure.

FIG. 6A is a plot of an adjusted pressure and adjusted ramp pressure, according to some implementations of the present disclosure.

FIG. 6B is a plot of an adjusted pressure response, according to some implementations of the present disclosure.

FIG. 7 is a process flow diagram of a method for optimizing one or more parameters of a respiratory therapy system, according to some implementations of the present disclosure.

FIG. 8 is a plot of an expiratory pressure relief setting with an initial value and a plot of the expiratory pressure relief setting with a recommended value, according to some implementations of the present disclosure.

FIG. 9 is a user device presenting recommended values of a plurality of parameters to a user, according to some implementations of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

The present disclosure is described with reference to the attached figures, where like reference numerals are used throughout the figures to designate similar or equivalent elements. The figures are not drawn to scale, and are provided merely to illustrate the instant disclosure. Several aspects of the disclosure are described below with reference to example applications for illustration.

Many individuals suffer from sleep-related and/or respiratory disorders. Examples of sleep-related and/or respiratory disorders include Periodic Limb Movement Disorder (PLMD), Restless Leg Syndrome (RLS), Sleep-Disordered Breathing (SDB), Obstructive Sleep Apnea (OSA), Central Sleep Apnea (CSA), other types of apneas, Cheyne-Stokes Respiration (CSR), respiratory insufficiency, Obesity Hyperventilation Syndrome (OHS), Chronic Obstructive Pulmonary Disease (COPD), Neuromuscular Disease (NMD), and chest wall disorders.

Many individuals suffer from sleep-related and/or respiratory disorders, such as Periodic Limb Movement Disorder (PLMD), Restless Leg Syndrome (RLS), Sleep-Disordered Breathing (SDB) such as Obstructive Sleep Apnea (OSA), Central Sleep Apnea (CSA) and other types of apneas, Respiratory Effort Related Arousal (RERA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), and chest wall disorders. Obstructive Sleep Apnea (OSA), a form of Sleep Disordered Breathing (SDB), is characterized by events including occlusion or obstruction of the upper air passage during sleep resulting from a combination of an abnormally small upper airway and the normal loss of muscle tone in the region of the tongue, soft palate, and posterior oropharyngeal wall. Central Sleep Apnea (CSA) is another form of sleep disordered breathing. CSA results when the brain temporarily stops sending signals to the muscles that control breathing. Other types of apneas include hypopnea, hyperpnea, and hypercapnia. Hypopnea is generally characterized by slow or shallow breathing caused by a narrowed airway, as opposed to a blocked airway. Hyperpnea is generally characterized by an increase depth and/or rate of breathing. Hypercapnia is generally characterized by elevated or excessive carbon dioxide in the bloodstream, typically caused by inadequate respiration. A Respiratory Effort Related Arousal (RERA) event is typically characterized by an increased respiratory effort for ten seconds or longer leading to arousal from sleep and which does not fulfill the criteria for an apnea or hypopnea event. RERAs are defined as a sequence of breaths characterized by increasing respiratory effort leading to an arousal from sleep, but which does not meet criteria for an apnea or hypopnea. These events must fulfil both of the following criteria: (1) a pattern of progressively more negative esophageal pressure, terminated by a sudden change in pressure to a less negative level and an arousal, and (2) the event lasts ten seconds or longer. In some implementations, a Nasal Cannula/Pressure Transducer System is adequate and reliable in the detection of RERAs. A RERA detector may be based on a real flow signal derived from a respiratory therapy device. For example, a flow limitation measure may be determined based on a flow signal. A measure of arousal may then be derived as a function of the flow limitation measure and a measure of sudden increase in ventilation. One such method is described in WO 2008/138040 and U.S. Pat. No. 9,358,353, assigned to ResMed Ltd., the disclosure of each of which is hereby incorporated by reference herein in their entireties.

Cheyne-Stokes Respiration (CSR) is a further form of SDB. CSR is a disorder of a patient's respiratory controller in which there are rhythmic alternating periods of waxing and waning ventilation known as CSR cycles. CSR is characterized by repetitive de-oxygenation and re-oxygenation of the arterial blood. OHS is defined as the combination of severe obesity and awake chronic hypercapnia, in the absence of other known causes for hypoventilation. Symptoms include dyspnea, morning headache and excessive daytime sleepiness. COPD encompasses any of a group of lower airway diseases that have certain characteristics in common, such as increased resistance to air movement, extended expiratory phase of respiration, and loss of the normal elasticity of the lung. NMD encompasses many diseases and ailments that impair the functioning of the muscles either directly via intrinsic muscle pathology, or indirectly via nerve pathology. Chest wall disorders are a group of thoracic deformities that result in inefficient coupling between the respiratory muscles and the thoracic cage.

Many of these disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that can occur when the individual is sleeping. A wide variety of types of data can be used to monitor the health of individuals having any of the above types of sleep-related and/or respiratory disorders (or other disorders).

The Apnea-Hypopnea Index (AHI) is an index used to indicate the severity of sleep apnea during a sleep session. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea.

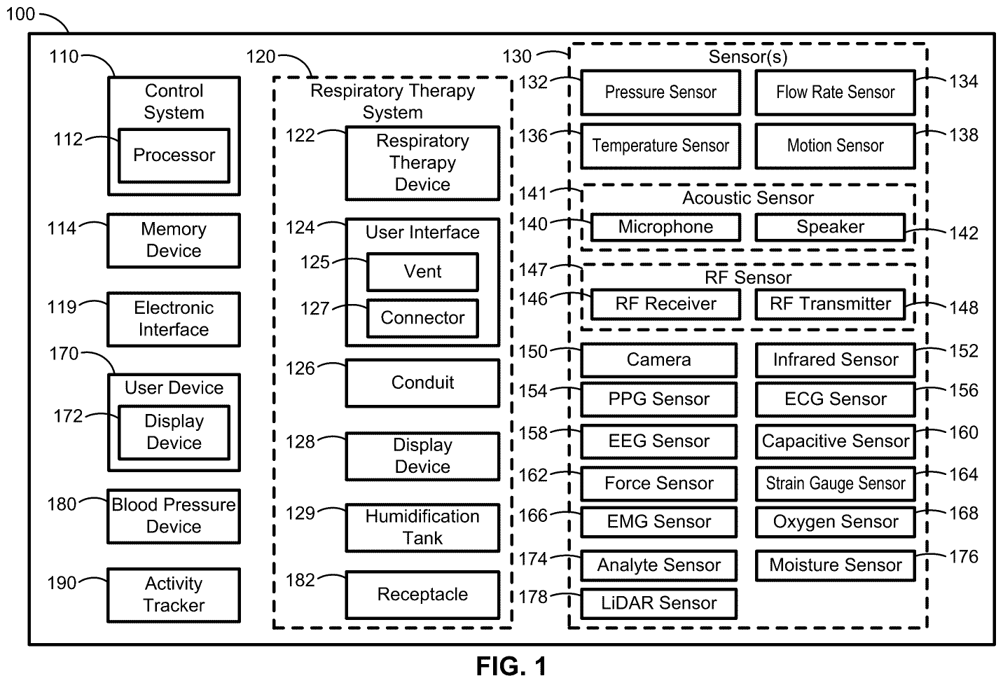

Referring to FIG. 1, a system 100, according to some implementations of the present disclosure, is illustrated. The system 100 includes a control system 110, a memory device 114, an electronic interface 119, one or more sensors 130, and optionally one or more user devices 170. In some implementations, the system 100 further includes a respiratory therapy system 120 (that includes a respiratory therapy device 122), a blood pressure device 180, an activity tracker 190, or any combination thereof. The system 100 can be used to optimize the values of one or more parameters of the respiratory therapy system.

The control system 110 includes one or more processors 112 (hereinafter, processor 112). The control system 110 is generally used to control (e.g., actuate) the various components of the system 100 and/or analyze data obtained and/or generated by the components of the system 100. The processor 112 can be a general or special purpose processor or microprocessor. While one processor 112 is shown in FIG. 1, the control system 110 can include any suitable number of processors (e.g., one processor, two processors, five processors, ten processors, etc.) that can be in a single housing, or located remotely from each other. The control system 110 (or any other control system) or a portion of the control system 110 such as the processor 112 (or any other processor(s) or portion(s) of any other control system), can be used to carry out one or more steps of any of the methods described and/or claimed herein. The control system 110 can be coupled to and/or positioned within, for example, a housing of the user device 170, and/or within a housing of one or more of the sensors 130. The control system 110 can be centralized (within one such housing) or decentralized (within two or more of such housings, which are physically distinct). In such implementations including two or more housings containing the control system 110, such housings can be located proximately and/or remotely from each other.

The memory device 114 stores machine-readable instructions thereon that are executable by the processor 112 of the control system 110. The memory device 114 can be any suitable computer readable storage device or media, such as, for example, a random or serial access memory device, a hard drive, a solid state drive, a flash memory device, etc. While one memory device 114 is shown in FIG. 1, the system 100 can include any suitable number of memory devices 114 (e.g., one memory device, two memory devices, five memory devices, ten memory devices, etc.). The memory device 114 can be coupled to and/or positioned within a housing of the respiratory therapy device 122 of the respiratory therapy system 120, within a housing of the user device 170, within a housing of one or more of the sensors 130, or any combination thereof. Like the control system 110, the memory device 114 can be centralized (within one such housing) or decentralized (within two or more of such housings, which are physically distinct).

In some implementations, the memory device 114 stores a user profile associated with the user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The demographic information can include, for example, information indicative of an age of the user, a gender of the user, a race of the user, a family medical history (such as a family history of insomnia or sleep apnea), an employment status of the user, an educational status of the user, a socioeconomic status of the user, or any combination thereof. The medical information can include, for example, information indicative of one or more medical conditions associated with the user, medication usage by the user, or both. The medical information data can further include a fall risk assessment associated with the user (e.g., a fall risk score using the Morse fall scale), a multiple sleep latency test (MSLT) result or score and/or a Pittsburgh Sleep Quality Index (PSQI) score or value. The self-reported user feedback can include information indicative of a self-reported subjective sleep score (e.g., poor, average, excellent), a self-reported subjective stress level of the user, a self-reported subjective fatigue level of the user, a self-reported subjective health status of the user, a recent life event experienced by the user, or any combination thereof.

The electronic interface 119 is configured to receive data (e.g., physiological data and/or acoustic data) from the one or more sensors 130 such that the data can be stored in the memory device 114 and/or analyzed by the processor 112 of the control system 110. The electronic interface 119 can communicate with the one or more sensors 130 using a wired connection or a wireless connection (e.g., using an RF communication protocol, a WiFi communication protocol, a Bluetooth communication protocol, an IR communication protocol, over a cellular network, over any other optical communication protocol, etc.). The electronic interface 119 can include an antenna, a receiver (e.g., an RF receiver), a transmitter (e.g., an RF transmitter), a transceiver, or any combination thereof. The electronic interface 119 can also include one more processors and/or one more memory devices that are the same as, or similar to, the processor 112 and the memory device 114 described herein. In some implementations, the electronic interface 119 is coupled to or integrated in the user device 170. In other implementations, the electronic interface 119 is coupled to or integrated (e.g., in a housing) with the control system 110 and/or the memory device 114.

As noted above, in some implementations, the system 100 optionally includes a respiratory therapy system 120 (also referred to as a respiratory pressure therapy system). The respiratory therapy system 120 can include a respiratory therapy device 122 (also referred to as a respiratory pressure device), a user interface 124 (also referred to as a mask or a patient interface), a conduit 126 (also referred to as a tube or an air circuit), a display device 128, a humidification tank 129, a receptacle 182, or any combination thereof. In some implementations, the control system 110, the memory device 114, the display device 128, one or more of the sensors 130, the humidification tank 129, and the receptacle 182 are part of the respiratory therapy device 122. Respiratory pressure therapy refers to the application of a supply of air to an entrance to a user's airways at a controlled target pressure that is nominally positive with respect to atmosphere throughout the user's breathing cycle (e.g., in contrast to negative pressure therapies such as the tank ventilator or cuirass). The respiratory therapy system 120 is generally used to treat individuals suffering from one or more sleep-related respiratory disorders (e.g., obstructive sleep apnea, central sleep apnea, or mixed sleep apnea), other respiratory disorders such as COPD, or other disorders leading to respiratory insufficiency, that may manifest either during sleep or wakefulness.

The respiratory therapy device 122 is generally used to generate pressurized air that is delivered to a user (e.g., using one or more motors (such as a blower motor) that drive one or more compressors). In some implementations, the respiratory therapy device 122 generates continuous constant air pressure that is delivered to the user. In other implementations, the respiratory therapy device 122 generates two or more predetermined pressures (e.g., a first predetermined air pressure and a second predetermined air pressure). In still other implementations, the respiratory therapy device 122 is configured to generate a variety of different air pressures within a predetermined range. For example, the respiratory therapy device 122 can deliver at least about 6 cm H₂O, at least about 10 cm H₂O, at least about 20 cm H₂O, between about 6 cm H₂O and about 10 cm H₂O, between about 7 cm H₂O and about 12 cm H₂O, etc. The respiratory therapy device 122 can also deliver pressurized air at a predetermined flow rate between, for example, about −20 L/min and about 150 L/min, while maintaining a positive pressure (relative to the ambient pressure). In some implementations, the control system 110, the memory device 114, the electronic interface 119, or any combination thereof can be coupled to and/or positioned within a housing of the respiratory therapy device 122.

The user interface 124 engages a portion of the user's face and delivers pressurized air from the respiratory therapy device 122 to the user's airway to aid in preventing the airway from narrowing and/or collapsing during sleep. This may also increase the user's oxygen intake during sleep. Depending upon the therapy to be applied, the user interface 124 may form a seal, for example, with a region or portion of the user's face, to facilitate the delivery of gas at a pressure at sufficient variance with ambient pressure to effect therapy, for example, at a positive pressure of about 10 cm H₂O relative to ambient pressure. For other forms of therapy, such as the delivery of oxygen, the user interface may not include a seal sufficient to facilitate delivery to the airways of a supply of gas at a positive pressure of about 10 cm H₂O.

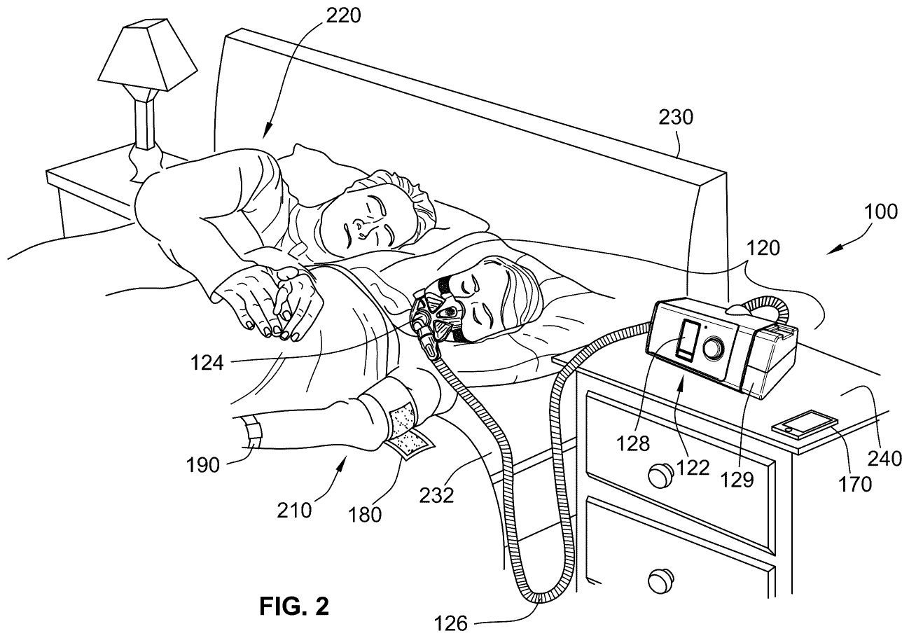

In some implementations, the user interface 124 is or includes a facial mask that covers the nose and mouth of the user (as shown, for example, in FIG. 2). Alternatively, the user interface 124 is or includes a nasal mask that provides air to the nose of the user or a nasal pillow mask that delivers air directly to the nostrils of the user. The user interface 124 can include a strap assembly that has a plurality of straps (e.g., including hook and loop fasteners) for positioning and/or stabilizing the user interface 124 on a portion of the user interface 124 on a desired location of the user (e.g., the face), and a conformal cushion (e.g., silicone, plastic, foam, etc.) that aids in providing an air-tight seal between the user interface 124 and the user. In some implementations, the user interface 124 may include a connector 127 and one or more vents 125. The one or more vents 125 can be used to permit the escape of carbon dioxide and other gases exhaled by the user. In other implementations, the user interface 124 includes a mouthpiece (e.g., a night guard mouthpiece molded to conform to the user's teeth, a mandibular repositioning device, etc.). In some implementations, the connector 127 is distinct from, but couplable to, the user interface 124 (and/or conduit 126). The connector 127 is configured to connect and fluidly couple the user interface 124 to the conduit 126.

The conduit 126 allows the flow of air between two components of a respiratory therapy system 120, such as the respiratory therapy device 122 and the user interface 124. In some implementations, there can be separate limbs of the conduit for inhalation and exhalation. In other implementations, a single limb conduit is used for both inhalation and exhalation. Generally, the respiratory therapy system 120 forms an air pathway that extends between a motor of the respiratory therapy device 122 and the user and/or the user's airway. Thus, the air pathway generally includes at least a motor of the respiratory therapy device 122, the user interface 124, and the conduit 126.

One or more of the respiratory therapy device 122, the user interface 124, the conduit 126, the display device 128, and the humidification tank 129 can contain one or more sensors (e.g., a pressure sensor, a flow rate sensor, or more generally any of the other sensors 130 described herein). These one or more sensors can be used, for example, to measure the air pressure and/or flow rate of pressurized air supplied by the respiratory therapy device 122.

The display device 128 is generally used to display image(s) including still images, video images, or both and/or information regarding the respiratory therapy device 122. For example, the display device 128 can provide information regarding the status of the respiratory therapy device 122 (e.g., whether the respiratory therapy device 122 is on/off, the pressure of the air being delivered by the respiratory therapy device 122, the temperature of the air being delivered by the respiratory therapy device 122, etc.) and/or other information (e.g., a sleep score or a therapy score (such as a myAir® score, such as described in WO 2016/061629 and US 2017/0311879, each of which is hereby incorporated by reference herein in its entirety), the current date/time, personal information for the user, a questionnaire for the user, etc.). In some implementations, the display device 128 acts as a human-machine interface (HMI) that includes a graphic user interface (GUI) configured to display the image(s) as an input interface. The display device 128 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory therapy device 122.

The humidification tank 129 is coupled to or integrated in the respiratory therapy device 122 and includes a reservoir of water that can be used to humidify the pressurized air delivered from the respiratory therapy device 122. The respiratory therapy device 122 can include a heater to heat the water in the humidification tank 129 in order to humidify the pressurized air provided to the user. Additionally, in some implementations, the conduit 126 can also include a heating element (e.g., coupled to and/or imbedded in the conduit 126) that heats the pressurized air delivered to the user. The humidification tank 129 can be fluidly coupled to a water vapor inlet of the air pathway and deliver water vapor into the air pathway via the water vapor inlet, or can be formed in-line with the air pathway as part of the air pathway itself. In other implementations, the respiratory therapy device 122 or the conduit 126 can include a waterless humidifier. The waterless humidifier can incorporate sensors that interface with other sensor positioned elsewhere in system 100.

In some implementations, the system 100 can be used to deliver at least a portion of a substance from the receptacle 182 to the air pathway of the user based at least in part on the physiological data, the sleep-related parameters, other data or information, or any combination thereof. Generally, modifying the delivery of the portion of the substance into the air pathway can include (i) initiating the delivery of the substance into the air pathway, (ii) ending the delivery of the portion of the substance into the air pathway, (iii) modifying an amount of the substance delivered into the air pathway, (iv) modifying a temporal characteristic of the delivery of the portion of the substance into the air pathway, (v) modifying a quantitative characteristic of the delivery of the portion of the substance into the air pathway, (vi) modifying any parameter associated with the delivery of the substance into the air pathway, or (vii) a combination of (i)-(vi).

Modifying the temporal characteristic of the delivery of the portion of the substance into the air pathway can include changing the rate at which the substance is delivered, starting and/or finishing at different times, continuing for different time periods, changing the time distribution or characteristics of the delivery, changing the amount distribution independently of the time distribution, etc. The independent time and amount variation ensures that, apart from varying the frequency of the release of the substance, one can vary the amount of substance released each time. In this manner, a number of different combination of release frequencies and release amounts (e.g., higher frequency but lower release amount, higher frequency and higher amount, lower frequency and higher amount, lower frequency and lower amount, etc.) can be achieved. Other modifications to the delivery of the portion of the substance into the air pathway can also be utilized.

The respiratory therapy system 120 can be used, for example, as a ventilator or a positive airway pressure (PAP) system, such as a continuous positive airway pressure (CPAP) system, an automatic positive airway pressure system (APAP), a bi-level or variable positive airway pressure system (BPAP or VPAP), or any combination thereof. The CPAP system delivers a predetermined air pressure (e.g., determined by a sleep physician) to the user. The APAP system automatically varies the air pressure delivered to the user based at least in part on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

Referring to FIG. 2, a portion of the system 100 (FIG. 1), according to some implementations, is illustrated. A user 210 of the respiratory therapy system 120 and a bed partner 220 are located in a bed 230 and are laying on a mattress 232. The user interface 124 (e.g., a full facial mask) can be worn by the user 210 during a sleep session. The user interface 124 is fluidly coupled and/or connected to the respiratory therapy device 122 via the conduit 126. In turn, the respiratory therapy device 122 delivers pressurized air to the user 210 via the conduit 126 and the user interface 124 to increase the air pressure in the throat of the user 210 to aid in preventing the airway from closing and/or narrowing during sleep. The respiratory therapy device 122 can include the display device 128, which can allow the user to interact with the respiratory therapy device 122. The respiratory therapy device 122 can also include the humidification tank 129, which stores the water used to humidify the pressurized air. The respiratory therapy device 122 can be positioned on a nightstand 240 that is directly adjacent to the bed 230 as shown in FIG. 2, or more generally, on any surface or structure that is generally adjacent to the bed 230 and/or the user 210. The user can also wear the blood pressure device 180 and the activity tracker 190 while lying on the mattress 232 in the bed 230.

Referring back to FIG. 1, the one or more sensors 130 of the system 100 include a pressure sensor 132, a flow rate sensor 134, temperature sensor 136, a motion sensor 138, a microphone 140, a speaker 142, a radio-frequency (RF) receiver 146, an RF transmitter 148, a camera 150, an infrared (IR) sensor 152, a photoplethysmogram (PPG) sensor 154, an electrocardiogram (ECG) sensor 156, an electroencephalography (EEG) sensor 158, a capacitive sensor 160, a force sensor 162, a strain gauge sensor 164, an electromyography (EMG) sensor 166, an oxygen sensor 168, an analyte sensor 174, a moisture sensor 176, a light detection and ranging (LiDAR) sensor 178, or any combination thereof. Generally, each of the one or sensors 130 are configured to output sensor data that is received and stored in the memory device 114 or one or more other memory devices. The sensors 130 can also include, an electrooculography (EOG) sensor, a peripheral oxygen saturation (SpO₂) sensor, a galvanic skin response (GSR) sensor, a carbon dioxide (CO₂) sensor, or any combination thereof.

While the one or more sensors 130 are shown and described as including each of the pressure sensor 132, the flow rate sensor 134, the temperature sensor 136, the motion sensor 138, the microphone 140, the speaker 142, the RF receiver 146, the RF transmitter 148, the camera 150, the IR sensor 152, the PPG sensor 154, the ECG sensor 156, the EEG sensor 158, the capacitive sensor 160, the force sensor 162, the strain gauge sensor 164, the EMG sensor 166, the oxygen sensor 168, the analyte sensor 174, the moisture sensor 176, and the LiDAR sensor 178, more generally, the one or more sensors 130 can include any combination and any number of each of the sensors described and/or shown herein.

The one or more sensors 130 can be used to generate, for example physiological data, acoustic data, or both, that is associated with a user of the respiratory therapy system 120 (such as the user 210 of FIG. 2), the respiratory therapy system 120, both the user and the respiratory therapy system 120, or other entities, objects, activities, etc. Physiological data generated by one or more of the sensors 130 can be used by the control system 110 to determine a sleep-wake signal associated with the user during the sleep session and one or more sleep-related parameters. The sleep-wake signal can be indicative of one or more sleep stages (sometimes referred to as sleep states), including sleep, wakefulness, relaxed wakefulness, micro-awakenings, or distinct sleep stages such as a rapid eye movement (REM) stage (which can include both a typical REM stage and an atypical REM stage), a first non-REM stage (often referred to as “N1”), a second non-REM stage (often referred to as “N2”), a third non-REM stage (often referred to as “N3”), or any combination thereof. Methods for determining sleep stages from physiological data generated by one or more of the sensors, such as sensors 130, are described in, for example, WO 2014/047310, U.S. Pat. Nos. 10,492,720, 10,660,563, US 2020/0337634, WO 2017/132726, WO 2019/122413, US 2021/0150873, WO 2019/122414, US 2020/0383580, each of which is hereby incorporated by reference herein in its entirety.

The sleep-wake signal can also be timestamped to indicate a time that the user enters the bed, a time that the user exits the bed, a time that the user attempts to fall asleep, etc. The sleep-wake signal can be measured one or more of the sensors 130 during the sleep session at a predetermined sampling rate, such as, for example, one sample per second, one sample per 30 seconds, one sample per minute, etc. Examples of the one or more sleep-related parameters that can be determined for the user during the sleep session based at least in part on the sleep-wake signal include a total time in bed, a total sleep time, a total wake time, a sleep onset latency, a wake-after-sleep-onset parameter, a sleep efficiency, a fragmentation index, an amount of time to fall asleep, a consistency of breathing rate, a fall asleep time, a wake time, a rate of sleep disturbances, a number of movements, or any combination thereof.

Physiological data and/or acoustic data generated by the one or more sensors 130 can also be used to determine a respiration signal associated with the user during a sleep session. The respiration signal is generally indicative of respiration or breathing of the user during the sleep session. The respiration signal can be indicative of, for example, a respiration rate, a respiration rate variability, an inspiration amplitude, an expiration amplitude, an inspiration-expiration amplitude ratio, an inspiration-expiration duration ratio, a number of events per hour, a pattern of events, pressure settings of the respiratory therapy device 122, or any combination thereof. The event(s) can include snoring, apneas, central apneas, obstructive apneas, mixed apneas, hypopneas, RERAs, a flow limitation (e.g., an event that results in the absence of the increase in flow despite an elevation in negative intrathoracic pressure indicating increased effort), a mask leak (e.g., from the user interface 124), a restless leg, a sleeping disorder, choking, an increased heart rate, a heart rate variation, labored breathing, an asthma attack, an epileptic episode, a seizure, a fever, a cough, a sneeze, a snore, a gasp, the presence of an illness such as the common cold or the flu, an elevated stress level, etc. Events can be detected by any means known in the art such as described in, for example, U.S. Pat. Nos. 5,245,995, 6,502,572, WO 2018/050913, WO 2020/104465, each of which is incorporated by reference herein in its entirety.

The pressure sensor 132 outputs pressure data that can be stored in the memory device 114 and/or analyzed by the processor 112 of the control system 110. In some implementations, the pressure sensor 132 is an air pressure sensor (e.g., barometric pressure sensor) that generates sensor data indicative of the respiration (e.g., inhaling and/or exhaling) of the user of the respiratory therapy system 120 and/or ambient pressure. In such implementations, the pressure sensor 132 can be coupled to or integrated in the respiratory therapy device 122. The pressure sensor 132 can be, for example, a capacitive sensor, an electromagnetic sensor, an inductive sensor, a resistive sensor, a piezoelectric sensor, a strain-gauge sensor, an optical sensor, a potentiometric sensor, or any combination thereof. In one example, the pressure sensor 132 can be used to determine a blood pressure of the user.

The flow rate sensor 134 outputs flow rate data that can be stored in the memory device 114 and/or analyzed by the processor 112 of the control system 110. In some implementations, the flow rate sensor 134 is used to determine an air flow rate from the respiratory therapy device 122, an air flow rate through the conduit 126, an air flow rate through the user interface 124, or any combination thereof. In such implementations, the flow rate sensor 134 can be coupled to or integrated in the respiratory therapy device 122, the user interface 124, or the conduit 126. The flow rate sensor 134 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof.

The temperature sensor 136 outputs temperature data that can be stored in the memory device 114 and/or analyzed by the processor 112 of the control system 110. In some implementations, the temperature sensor 136 generates temperatures data indicative of a core body temperature of the user, a skin temperature of the user 210, a temperature of the air flowing from the respiratory therapy device 122 and/or through the conduit 126, a temperature in the user interface 124, an ambient temperature, or any combination thereof. The temperature sensor 136 can be, for example, a thermocouple sensor, a thermistor sensor, a silicon band gap temperature sensor or semiconductor-based sensor, a resistance temperature detector, or any combination thereof.

The motion sensor 138 outputs motion data that can be stored in the memory device 114 and/or analyzed by the processor 112 of the control system 110. The motion sensor 138 can be used to detect movement of the user during the sleep session, and/or detect movement of any of the components of the respiratory therapy system 120, such as the respiratory therapy device 122, the user interface 124, or the conduit 126. The motion sensor 138 can include one or more inertial sensors, such as accelerometers, gyroscopes, and magnetometers. The motion sensor 138 can be used to detect motion or acceleration associated with arterial pulses, such as pulses in or around the face of the user and proximal to the user interface 124, and configured to detect features of the pulse shape, speed, amplitude, or volume. In some implementations, the motion sensor 138 alternatively or additionally generates one or more signals representing bodily movement of the user, from which may be obtained a signal representing a sleep state of the user; for example, via a respiratory movement of the user.

The microphone 140 outputs acoustic data that can be stored in the memory device 114 and/or analyzed by the processor 112 of the control system 110. The acoustic data generated by the microphone 140 is reproducible as one or more sound(s) during a sleep session (e.g., sounds from the user) to determine (e.g., using the control system 110) one or more sleep-related parameters, as described in further detail herein. The acoustic data from the microphone 140 can also be used to identify (e.g., using the control system 110) an event experienced by the user during the sleep session, as described in further detail herein. In other implementations, the acoustic data from the microphone 140 is representative of noise associated with the respiratory therapy system 120. In some implementations, the acoustic data from the microphone 140 can be analyzed to detect the presence of liquid in the respiratory therapy system 120, in particular in the user interface 124 and/or the conduit 126, as explained in further detail herein. In some implementations, the system 100 includes a plurality of microphones (e.g., two or more microphones and/or an array of microphones with beamforming) such that sound data generated by each of the plurality of microphones can be used to discriminate the sound data generated by another of the plurality of microphones. The microphone 140 can be coupled to or integrated in the respiratory therapy system 120 (or the system 100) generally in any configuration. For example, the microphone 140 can be disposed inside the respiratory therapy device 122, the user interface 124, the conduit 126, or other components. The microphone 140 can also be positioned adjacent to or coupled to the outside of the respiratory therapy device 122, the outside of the user interface 124, the outside of the conduit 126, or outside of any other components. The microphone 140 could also be a component of the user device 170 (e.g., the microphone 140 is a microphone of a smart phone). The microphone 140 can be integrated into the user interface 124, the conduit 126, the respiratory therapy device 122, or any combination thereof. In general, the microphone 140 can be located at any point within or adjacent to the air pathway of the respiratory therapy system 120, which includes at least the motor of the respiratory therapy device 122, the user interface 124, and the conduit 126. Thus, the air pathway can also be referred to as the acoustic pathway.

The speaker 142 outputs sound waves that are typically audible to the user. In one or more implementations, the sound waves can be audible to a user of the system 100 or inaudible to the user of the system (e.g., ultrasonic sound waves). The speaker 142 can be used, for example, as an alarm clock or to play an alert or message to the user (e.g., in response to an event). In some implementations, the speaker 142 can be used to communicate the acoustic data generated by the microphone 140 to the user. The speaker 142 can be coupled to or integrated in the respiratory therapy device 122, the user interface 124, the conduit 126, or the user device 170.

The microphone 140 and the speaker 142 can be used as separate devices. In some implementations, the microphone 140 and the speaker 142 can be combined into an acoustic sensor 141 (e.g., a SONAR sensor), as described in, for example, WO 2018/050913 and WO 2020/104465, each of which is hereby incorporated by reference herein in its entirety. In such implementations, the speaker 142 generates or emits sound waves at a predetermined interval and/or frequency, and the microphone 140 detects the reflections of the emitted sound waves from the speaker 142. The sound waves generated or emitted by the speaker 142 have a frequency that is not audible to the human ear (e.g., below 20 Hz or above around 18 kHz) so as not to disturb the sleep of the user or a bed partner of the user (such as bed partner 220 in FIG. 2). Based at least in part on the data from the microphone 140 and/or the speaker 142, the control system 110 can determine a location of the user and/or one or more of the sleep-related parameters described in herein, such as, for example, a respiration signal, a respiration rate, an inspiration amplitude, an expiration amplitude, an inspiration-expiration ratio, a number of events per hour, a pattern of events, a sleep stage, pressure settings of the respiratory therapy device 122, a mouth leak status, or any combination thereof. In this context, a SONAR sensor may be understood to concern an active acoustic sensing, such as by generating/transmitting ultrasound or low frequency ultrasound sensing signals (e.g., in a frequency range of about 17-23 kHz, 18-22 kHz, or 17-18 kHz, for example), through the air. Such a system may be considered in relation to WO 2018/050913 and WO 2020/104465 mentioned above. In some implementations, the speaker 142 is a bone conduction speaker. In some implementations, the one or more sensors 130 include (i) a first microphone that is the same or similar to the microphone 140, and is integrated into the acoustic sensor 141 and (ii) a second microphone that is the same as or similar to the microphone 140, but is separate and distinct from the first microphone that is integrated into the acoustic sensor 141.

The RF transmitter 148 generates and/or emits radio waves having a predetermined frequency and/or a predetermined amplitude (e.g., within a high frequency band, within a low frequency band, long wave signals, short wave signals, etc.). The RF receiver 146 detects the reflections of the radio waves emitted from the RF transmitter 148, and this data can be analyzed by the control system 110 to determine a location of the user and/or one or more of the sleep-related parameters described herein. An RF receiver (either the RF receiver 146 and the RF transmitter 148 or another RF pair) can also be used for wireless communication between the control system 110, the respiratory therapy device 122, the one or more sensors 130, the user device 170, or any combination thereof. While the RF receiver 146 and RF transmitter 148 are shown as being separate and distinct elements in FIG. 1, in some implementations, the RF receiver 146 and RF transmitter 148 are combined as a part of an RF sensor 147 (e.g., a RADAR sensor). In some such implementations, the RF sensor 147 includes a control circuit. The specific format of the RF communication could be WiFi, Bluetooth, etc.

In some implementations, the RF sensor 147 is a part of a mesh system. One example of a mesh system is a WiFi mesh system, which can include mesh nodes, mesh router(s), and mesh gateway(s), each of which can be mobile/movable or fixed. In such implementations, the WiFi mesh system includes a WiFi router and/or a WiFi controller and one or more satellites (e.g., access points), each of which include an RF sensor that the is the same as, or similar to, the RF sensor 147. The WiFi router and satellites continuously communicate with one another using WiFi signals. The WiFi mesh system can be used to generate motion data based at least in part on changes in the WiFi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 150 outputs image data reproducible as one or more images (e.g., still images, video images, thermal images, or a combination thereof) that can be stored in the memory device 114. The image data from the camera 150 can be used by the control system 110 to determine one or more of the sleep-related parameters described herein. For example, the image data from the camera 150 can be used to identify a location of the user, to determine a time when the user enters the user's bed (such as bed 230 in FIG. 2), and to determine a time when the user exits the bed 230. The camera 150 can also be used to track eye movements, pupil dilation (if one or both of the user's eyes are open), blink rate, or any changes during REM sleep. The camera 150 can also be used to track the position of the user, which can impact the duration and/or severity of apneic episodes in users with positional obstructive sleep apnea.

The IR sensor 152 outputs infrared image data reproducible as one or more infrared images (e.g., still images, video images, or both) that can be stored in the memory device 114. The infrared data from the IR sensor 152 can be used to determine one or more sleep-related parameters during the sleep session, including a temperature of the user and/or movement of the user. The IR sensor 152 can also be used in conjunction with the camera 150 when measuring the presence, location, and/or movement of the user. The IR sensor 152 can detect infrared light having a wavelength between about 700 nm and about 1 mm, for example, while the camera 150 can detect visible light having a wavelength between about 380 nm and about 740 nm.

The IR sensor 152 outputs infrared image data reproducible as one or more infrared images (e.g., still images, video images, or both) that can be stored in the memory device 114. The infrared data from the IR sensor 152 can be used to determine one or more sleep-related parameters during the sleep session, including a temperature of the user and/or movement of the user. The IR sensor 152 can also be used in conjunction with the camera 150 when measuring the presence, location, and/or movement of the user. The IR sensor 152 can detect infrared light having a wavelength between about 700 nm and about 1 mm, for example, while the camera 150 can detect visible light having a wavelength between about 380 nm and about 740 nm.

The PPG sensor 154 outputs physiological data associated with the user that can be used to determine one or more sleep-related parameters, such as, for example, a heart rate, a heart rate pattern, a heart rate variability, a cardiac cycle, respiration rate, an inspiration amplitude, an expiration amplitude, an inspiration-expiration ratio, estimated blood pressure parameter(s), or any combination thereof. The PPG sensor 154 can be worn by the user, embedded in clothing and/or fabric that is worn by the user, embedded in and/or coupled to the user interface 124 and/or its associated headgear (e.g., straps, etc.), etc.

The ECG sensor 156 outputs physiological data associated with electrical activity of the heart of the user. In some implementations, the ECG sensor 156 includes one or more electrodes that are positioned on or around a portion of the user during the sleep session. The physiological data from the ECG sensor 156 can be used, for example, to determine one or more of the sleep-related parameters described herein.

The EEG sensor 158 outputs physiological data associated with electrical activity of the brain of the user. In some implementations, the EEG sensor 158 includes one or more electrodes that are positioned on or around the scalp of the user during the sleep session. The physiological data from the EEG sensor 158 can be used, for example, to determine a sleep stage of the user at any given time during the sleep session. In some implementations, the EEG sensor 158 can be integrated in the user interface 124 and/or the associated headgear (e.g., straps, etc.).

The capacitive sensor 160, the force sensor 162, and the strain gauge sensor 164 output data that can be stored in the memory device 114 and used by the control system 110 to determine one or more of the sleep-related parameters described herein. The EMG sensor 166 outputs physiological data associated with electrical activity produced by one or more muscles. The oxygen sensor 168 outputs oxygen data indicative of an oxygen concentration of gas (e.g., in the conduit 126 or at the user interface 124). The oxygen sensor 168 can be, for example, an ultrasonic oxygen sensor, an electrical oxygen sensor, a chemical oxygen sensor, an optical oxygen sensor, or any combination thereof. In some implementations, the one or more sensors 130 also include a galvanic skin response (GSR) sensor, a blood flow sensor, a respiration sensor, a pulse sensor, a sphygmomanometer sensor, an oximetry sensor, or any combination thereof.

The analyte sensor 174 can be used to detect the presence of an analyte in the exhaled breath of the user. The data output by the analyte sensor 174 can be stored in the memory device 114 and used by the control system 110 to determine the identity and concentration of any analytes in the user's breath. In some implementations, the analyte sensor 174 is positioned near a mouth of the user to detect analytes in breath exhaled from the user's mouth. For example, when the user interface 124 is a facial mask that covers the nose and mouth of the user, the analyte sensor 174 can be positioned within the facial mask to monitor the user mouth breathing. In other implementations, such as when the user interface 124 is a nasal mask or a nasal pillow mask, the analyte sensor 174 can be positioned near the nose of the user to detect analytes in breath exhaled through the user's nose. In still other implementations, the analyte sensor 174 can be positioned near the user's mouth when the user interface 124 is a nasal mask or a nasal pillow mask. In this implementation, the analyte sensor 174 can be used to detect whether any air is inadvertently leaking from the user's mouth. In some implementations, the analyte sensor 174 is a volatile organic compound (VOC) sensor that can be used to detect carbon-based chemicals or compounds, such as carbon dioxide. In some implementations, the analyte sensor 174 can also be used to detect whether the user is breathing through their nose or mouth. For example, if the data output by an analyte sensor 174 positioned near the mouth of the user or within the facial mask (in implementations where the user interface 124 is a facial mask) detects the presence of an analyte, the control system 110 can use this data as an indication that the user is breathing through their mouth.

The moisture sensor 176 outputs data that can be stored in the memory device 114 and used by the control system 110. The moisture sensor 176 can be used to detect moisture in various areas surrounding the user (e.g., inside the conduit 126 or the user interface 124, near the user's face, near the connection between the conduit 126 and the user interface 124, near the connection between the conduit 126 and the respiratory therapy device 122, etc.). Thus, in some implementations, the moisture sensor 176 can be coupled to or integrated into the user interface 124 or in the conduit 126 to monitor the humidity of the pressurized air from the respiratory therapy device 122. In other implementations, the moisture sensor 176 is placed near any area where moisture levels need to be monitored. The moisture sensor 176 can also be used to monitor the humidity of the ambient environment surrounding the user, for example the air inside the user's bedroom. The moisture sensor 176 can also be used to track the user's biometric response to environmental changes.

One or more LiDAR sensors 178 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 178 can measure and map an area extending 5 meters or more away from the sensor. The LiDAR data can be fused with point cloud data estimated by an electromagnetic RADAR sensor, for example. The LiDAR sensor 178 may also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

While shown separately in FIG. 1, any combination of the one or more sensors 130 can be integrated in and/or coupled to any one or more of the components of the system 100, including the respiratory therapy device 122, the user interface 124, the conduit 126, the humidification tank 129, the control system 110, the user device 170, or any combination thereof. For example, the acoustic sensor 141 and/or the RF sensor 147 can be integrated in and/or coupled to the user device 170. In such implementations, the user device 170 can be considered a secondary device that generates additional or secondary data for use by the system 100 (e.g., the control system 110) according to some aspects of the present disclosure. In some implementations, the pressure sensor 132 and/or the flow rate sensor 134 are integrated into and/or coupled to the respiratory therapy device 122. In some implementations, at least one of the one or more sensors 130 is not coupled to the respiratory therapy device 122, the control system 110, or the user device 170, and is positioned generally adjacent to the user during the sleep session (e.g., positioned on or in contact with a portion of the user, worn by the user, coupled to or positioned on the nightstand, coupled to the mattress, coupled to the ceiling, etc.). More generally, the one or more sensors 130 can be positioned at any suitable location relative to the user such that the one or more sensors 130 can generate physiological data associated with the user and/or the bed partner 220 during one or more sleep session.

The data from the one or more sensors 130 can be analyzed to determine one or more sleep-related parameters, which can include a respiration signal, a respiration rate, a respiration pattern, an inspiration amplitude, an expiration amplitude, an inspiration-expiration ratio, an occurrence of one or more events, a number of events per hour, a pattern of events, an average duration of events, a range of event durations, a ratio between the number of different events, a sleep stage, an apnea-hypopnea index (AHI), or any combination thereof. The one or more events can include snoring, apneas, central apneas, obstructive apneas, mixed apneas, hypopneas, an intentional user interface leak, an unintentional user interface leak, a mouth leak, a cough, a restless leg, a sleeping disorder, choking, an increased heart rate, labored breathing, an asthma attack, an epileptic episode, a seizure, increased blood pressure, hyperventilation, or any combination thereof. Many of these sleep-related parameters are physiological parameters, although some of the sleep-related parameters can be considered to be non-physiological parameters. Other types of physiological and non-physiological parameters can also be determined, either from the data from the one or more sensors 130, or from other types of data.

The user device 170 includes a display device 172. The user device 170 can be, for example, a mobile device such as a smart phone, a tablet, a laptop, a gaming console, a smart watch, or the like. Alternatively, the user device 170 can be an external sensing system, a television (e.g., a smart television) or another smart home device (e.g., a smart speaker(s) such as Google Home®, Google Nest®, Amazon Echo®, Amazon Echo Show®, Alexa®-enabled devices, etc.). In some implementations, the user device 170 is a wearable device (e.g., a smart watch). The display device 172 is generally used to display image(s) including still images, video images, or both. In some implementations, the display device 172 acts as a human-machine interface (HMI) that includes a graphic user interface (GUI) configured to display the image(s) and an input interface. The display device 172 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 170. In some implementations, one or more user devices 170 can be used by and/or included in the system 100.

The blood pressure device 180 is generally used to aid in generating physiological data for determining one or more blood pressure measurements associated with a user. The blood pressure device 180 can include at least one of the one or more sensors 130 to measure, for example, a systolic blood pressure component and/or a diastolic blood pressure component.

In some implementations, the blood pressure device 180 is a sphygmomanometer including an inflatable cuff that can be worn by a user and a pressure sensor (e.g., the pressure sensor 132 described herein). For example, as shown in the example of FIG. 2, the blood pressure device 180 can be worn on an upper arm of the user. In such implementations where the blood pressure device 180 is a sphygmomanometer, the blood pressure device 180 also includes a pump (e.g., a manually operated bulb) for inflating the cuff. In some implementations, the blood pressure device 180 is coupled to the respiratory therapy device 122 of the respiratory therapy system 120, which in turn delivers pressurized air to inflate the cuff. More generally, the blood pressure device 180 can be communicatively coupled with, and/or physically integrated in (e.g., within a housing), the control system 110, the memory device 114, the respiratory therapy system 120, the user device 170, and/or the activity tracker 190.

The activity tracker 190 is generally used to aid in generating physiological data for determining an activity measurement associated with the user. The activity measurement can include, for example, a number of steps, a distance traveled, a number of steps climbed, a duration of physical activity, a type of physical activity, an intensity of physical activity, time spent standing, a respiration rate, an average respiration rate, a resting respiration rate, a maximum respiration rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), or any combination thereof. The activity tracker 190 includes one or more of the sensors 130 described herein, such as, for example, the motion sensor 138 (e.g., one or more accelerometers and/or gyroscopes), the PPG sensor 154, and/or the ECG sensor 156.

In some implementations, the activity tracker 190 is a wearable device that can be worn by the user, such as a smartwatch, a wristband, a ring, or a patch. For example, referring to FIG. 2, the activity tracker 190 is worn on a wrist of the user. The activity tracker 190 can also be coupled to or integrated a garment or clothing that is worn by the user. Alternatively, still, the activity tracker 190 can also be coupled to or integrated in (e.g., within the same housing) the user device 170. More generally, the activity tracker 190 can be communicatively coupled with, or physically integrated in (e.g., within a housing), the control system 110, the memory device 114, the respiratory therapy system 120, the user device 170, and/or the blood pressure device 180.

While the control system 110 and the memory device 114 are described and shown in FIG. 1 as being a separate and distinct component of the system 100, in some implementations, the control system 110 and/or the memory device 114 are integrated in the user device 170 and/or the respiratory therapy device 122. Alternatively, in some implementations, the control system 110 or a portion thereof (e.g., the processor 112) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

While system 100 is shown as including all of the components described above, more or fewer components can be included in a system for determining a length of a conduit, according to implementations of the present disclosure. For example, a first alternative system includes the control system 110, the memory device 114, and at least one of the one or more sensors 130. As another example, a second alternative system includes the control system 110, the memory device 114, at least one of the one or more sensors 130, and the user device 170. As yet another example, a third alternative system includes the control system 110, the memory device 114, the respiratory therapy system 120, at least one of the one or more sensors 130, and the user device 170. As a further example, a fourth alternative system includes the control system 110, the memory device 114, the respiratory therapy system 120, at least one of the one or more sensors 130, the user device 170, and the blood pressure device 180 and/or activity tracker 190. Thus, various systems for modifying pressure settings can be formed using any portion or portions of the components shown and described herein and/or in combination with one or more other components.

Referring again to FIG. 2, in some implementations, the control system 110, the memory device 114, any of the one or more sensors 130, or a combination thereof can be located on and/or in any surface and/or structure that is generally adjacent to the bed 230 and/or the user 210. For example, in some implementations, at least one of the one or more sensors 130 can be located at a first position on and/or in one or more components of the respiratory therapy system 120 adjacent to the bed 230 and/or the user 210. The one or more sensors 130 can be coupled to the respiratory therapy system 120, the user interface 124, the conduit 126, the display device 128, the humidification tank 129, or a combination thereof.

Alternatively, or additionally, at least one of the one or more sensors 130 can be located at a second position on and/or in the bed 230 (e.g., the one or more sensors 130 are coupled to and/or integrated in the bed 230). Further, alternatively or additionally, at least one of the one or more sensors 130 can be located at a third position on and/or in the mattress 232 that is adjacent to the bed 230 and/or the user 210 (e.g., the one or more sensors 130 are coupled to and/or integrated in the mattress 232). Alternatively, or additionally, at least one of the one or more sensors 130 can be located at a fourth position on and/or in a pillow that is generally adjacent to the bed 230 and/or the user 210.

Alternatively, or additionally, at least one of the one or more sensors 130 can be located at a fifth position on and/or in the nightstand 240 that is generally adjacent to the bed 230 and/or the user 210. Alternatively, or additionally, at least one of the one or more sensors 130 can be located at a sixth position such that the at least one of the one or more sensors 130 are coupled to and/or positioned on the user 210 (e.g., the one or more sensors 130 are embedded in or coupled to fabric, clothing, and/or a smart device worn by the user 210). More generally, at least one of the one or more sensors 130 can be positioned at any suitable location relative to the user 210 such that the one or more sensors 130 can generate sensor data associated with the user 210.

In some implementations, a primary sensor, such as the microphone 140, is configured to generate acoustic data associated with the user 210 during a sleep session. The acoustic data can be based on, for example, acoustic signals in the conduit 126 of the respiratory therapy system 120. For example, one or more microphones (the same as, or similar to, the microphone 140 of FIG. 1) can be integrated in and/or coupled to (i) a circuit board of the respiratory therapy device 122, (ii) the conduit 126, (iii) a connector between components of the respiratory therapy system 120, (iv) the user interface 124, (v) a headgear (e.g., straps) associated with the user interface, or (vi) a combination thereof. In some implementations, the microphone 140 is in fluid communication with the airflow pathway (e.g., an airflow pathway between the flow generator/motor and the distal end of the conduit). By fluid communication, it is intended to also include configurations wherein the microphone is in acoustic communication with the airflow pathway without being in direct or physical contact with the airflow. For example, in some implementations, the microphone is positioned on a circuit board and in fluid communication, optionally via a duct sealed by a membrane, to the airflow pathway.

In some implementations, one or more secondary sensors may be used in addition to the primary sensor to generate additional data. In some such implementations, the one or more secondary sensors include: a microphone (e.g., the microphone 140 of the system 100), a flow rate sensor (e.g., the flow rate sensor 134 of the system 100), a pressure sensor (e.g., the pressure sensor 132 of the system 100), a temperature sensor (e.g., the temperature sensor 136 of the system 100), a camera (e.g., the camera 150 of the system 100), a vane sensor (VAF), a hot wire sensor (MAF), a cold wire sensor, a laminar flow sensor, an ultrasonic sensor, an inertial sensor, or a combination thereof.

Additionally, or alternatively, one or more microphones (the same as, or similar to, the microphone 140 of FIG. 1) can be integrated in and/or coupled to a co-located smart device, such as the user device 170, a TV, a watch (e.g., a mechanical watch or another smart device worn by the user), a pendant, the mattress 232, the bed 230, beddings positioned on the bed 230, the pillow, a speaker (e.g., the speaker 142 of FIG. 1), a radio, a tablet device, a waterless humidifier, or a combination thereof. A co-located smart device can be any smart device that is within range for detecting sounds emitted by the user, the respiratory therapy system 120, and/or any portion of the system 100. In some implementations, the co-located smart device is a smart device that is in the same room as the user during the sleep session.

Additionally, or alternatively, in some implementations, one or more microphones (the same as, or similar to, the microphone 140 of FIG. 1) can be remote from the system 100 (FIG. 1) and/or the user 210 (FIG. 2), so long as there is an air passage allowing acoustic signals to travel to the one or more microphones. For example, the one or more microphones can be in a different room from the room containing the system 100.

As used herein, a sleep session can be defined in a number of ways based at least in part on, for example, an initial start time and an end time. In some implementations, a sleep session is a duration where the user is asleep, that is, the sleep session has a start time and an end time, and during the sleep session, the user does not wake until the end time. That is, any period of the user being awake is not included in a sleep session. From this first definition of sleep session, if the user wakes ups and falls asleep multiple times in the same night, each of the sleep intervals separated by an awake interval is a sleep session.

Alternatively, in some implementations, a sleep session has a start time and an end time, and during the sleep session, the user can wake up, without the sleep session ending, so long as a continuous duration that the user is awake is below an awake duration threshold. The awake duration threshold can be defined as a percentage of a sleep session. The awake duration threshold can be, for example, about twenty percent of the sleep session, about fifteen percent of the sleep session duration, about ten percent of the sleep session duration, about five percent of the sleep session duration, about two percent of the sleep session duration, etc., or any other threshold percentage. In some implementations, the awake duration threshold is defined as a fixed amount of time, such as, for example, about one hour, about thirty minutes, about fifteen minutes, about ten minutes, about five minutes, about two minutes, etc., or any other amount of time.

In some implementations, a sleep session is defined as the entire time between the time in the evening at which the user first entered the bed, and the time the next morning when user last left the bed. Put another way, a sleep session can be defined as a period of time that begins on a first date (e.g., Monday, Jan. 6, 2020) at a first time (e.g., 10:00 PM), that can be referred to as the current evening, when the user first enters a bed with the intention of going to sleep (e.g., not if the user intends to first watch television or play with a smart phone before going to sleep, etc.), and ends on a second date (e.g., Tuesday, Jan. 7, 2020) at a second time (e.g., 7:00 AM), that can be referred to as the next morning, when the user first exits the bed with the intention of not going back to sleep that next morning.

In some implementations, the user can manually define the beginning of a sleep session and/or manually terminate a sleep session. For example, the user can select (e.g., by clicking or tapping) one or more user-selectable element that is displayed on the display device 172 of the user device 170 (FIG. 1) to manually initiate or terminate the sleep session.

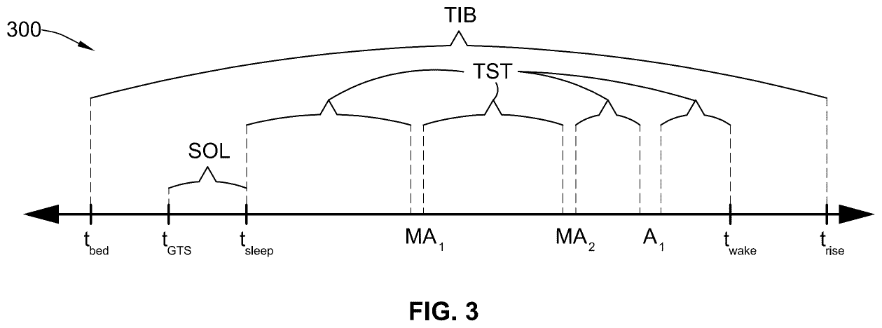

Referring to FIG. 3, an exemplary timeline 300 for a sleep session is illustrated. The timeline 300 includes an enter bed time (t_{bed}), a go-to-sleep time (t_{GTS}), an initial sleep time (tₛₗₑₑₚ), a first micro-awakening MA₁, a second micro-awakening MA₂, an awakening A, a wake-up time (t_{wake}), and a rising time (tᵣᵢₛₑ).

The enter bed time t_{bed} is associated with the time that the user initially enters the bed (e.g., bed 230 in FIG. 2) prior to falling asleep (e.g., when the user lies down or sits in the bed). The enter bed time t_{bed} can be identified based at least in part on a bed threshold duration to distinguish between times when the user enters the bed for sleep and when the user enters the bed for other reasons (e.g., to watch TV). For example, the bed threshold duration can be at least about 10 minutes, at least about 20 minutes, at least about 30 minutes, at least about 45 minutes, at least about 1 hour, at least about 2 hours, etc. While the enter bed time t_{bed} is described herein in reference to a bed, more generally, the enter time t_{bed} can refer to the time the user initially enters any location for sleeping (e.g., a couch, a chair, a sleeping bag, etc.).

The go-to-sleep time (GTS) is associated with the time that the user initially attempts to fall asleep after entering the bed (t_{bed}). For example, after entering the bed, the user may engage in one or more activities to wind down prior to trying to sleep (e.g., reading, watching TV, listening to music, using the user device 170, etc.). The initial sleep time (tₛₗₑₑₚ) is the time that the user initially falls asleep. For example, the initial sleep time (tₛₗₑₑₚ) can be the time that the user initially enters the first non-REM sleep stage.

The wake-up time t_{wake} is the time associated with the time when the user wakes up without going back to sleep (e.g., as opposed to the user waking up in the middle of the night and going back to sleep). The user may experience one of more unconscious microawakenings (e.g., microawakenings MA₁ and MA₂) having a short duration (e.g., 5 seconds, 10 seconds, 30 seconds, 1 minute, etc.) after initially falling asleep. In contrast to the wake-up time t_{wake}, the user goes back to sleep after each of the microawakenings MA₁ and MA₂. Similarly, the user may have one or more conscious awakenings (e.g., awakening A) after initially falling asleep (e.g., getting up to go to the bathroom, attending to children or pets, sleep walking, etc.). However, the user goes back to sleep after the awakening A. Thus, the wake-up time t_{wake} can be defined, for example, based at least in part on a wake threshold duration (e.g., the user is awake for at least 15 minutes, at least 20 minutes, at least 30 minutes, at least 1 hour, etc.).

Similarly, the rising time tᵣᵢₛₑ is associated with the time when the user exits the bed and stays out of the bed with the intent to end the sleep session (e.g., as opposed to the user getting up during the night to go to the bathroom, to attend to children or pets, sleep walking, etc.). In other words, the rising time tᵣᵢₛₑ is the time when the user last leaves the bed without returning to the bed until a next sleep session (e.g., the following evening). Thus, the rising time tᵣᵢₛₑ can be defined, for example, based at least in part on a rise threshold duration (e.g., the user has left the bed for at least 15 minutes, at least 20 minutes, at least 30 minutes, at least 1 hour, etc.). The enter bed time t_{bed} time for a second, subsequent sleep session can also be defined based at least in part on a rise threshold duration (e.g., the user has left the bed for at least 4 hours, at least 6 hours, at least 8 hours, at least 12 hours, etc.).

As described above, the user may wake up and get out of bed one more times during the night between the initial t_{bed} and the final tᵣᵢₛₑ. In some implementations, the final wake-up time t_{wake} and/or the final rising time tᵣᵢₛₑ that are identified or determined based at least in part on a predetermined threshold duration of time subsequent to an event (e.g., falling asleep or leaving the bed). Such a threshold duration can be customized for the user. For a standard user which goes to bed in the evening, then wakes up and goes out of bed in the morning any period (between the user waking up (t_{wake}) or raising up (tᵣᵢₛₑ), and the user either going to bed (t_{bed}), going to sleep (t_{GTS}) or falling asleep (tₛₗₑₑₚ) of between about 12 and about 18 hours can be used. For users that spend longer periods of time in bed, shorter threshold periods may be used (e.g., between about 8 hours and about 14 hours). The threshold period may be initially selected and/or later adjusted based at least in part on the system monitoring the user's sleep behavior.

The total time in bed (TIB) is the duration of time between the time enter bed time t_{bed} and the rising time tᵣᵢₛₑ. The total sleep time (TST) is associated with the duration between the initial sleep time and the wake-up time, excluding any conscious or unconscious awakenings and/or micro-awakenings therebetween. Generally, the total sleep time (TST) will be shorter than the total time in bed (TIB) (e.g., one minute short, ten minutes shorter, one hour shorter, etc.). For example, referring to the timeline 300 of FIG. 3, the total sleep time (TST) spans between the initial sleep time tₛₗₑₑₚ and the wake-up time t_{wake}, but excludes the duration of the first micro-awakening MA₁, the second micro-awakening MA₂, and the awakening A. As shown, in this example, the total sleep time (TST) is shorter than the total time in bed (TIB).

In some implementations, the total sleep time (TST) can be defined as a persistent total sleep time (PTST). In such implementations, the persistent total sleep time excludes a predetermined initial portion or period of the first non-REM stage (e.g., light sleep stage). For example, the predetermined initial portion can be between about 30 seconds and about 20minutes, between about 1 minute and about 10 minutes, between about 3 minutes and about 5 minutes, etc. The persistent total sleep time is a measure of sustained sleep, and smooths the sleep-wake hypnogram. For example, when the user is initially falling asleep, the user may be in the first non-REM stage for a very short time (e.g., about 30 seconds), then back into the wakefulness stage for a short period (e.g., one minute), and then goes back to the first non-REM stage. In this example, the persistent total sleep time excludes the first instance (e.g., about 30 seconds) of the first non-REM stage.

In some implementations, the sleep session is defined as starting at the enter bed time (t_{bed}) and ending at the rising time (tᵣᵢₛₑ), i.c., the sleep session is defined as the total time in bed (TIB). In some implementations, a sleep session is defined as starting at the initial sleep time (tₛₗₑₑₚ) and ending at the wake-up time (t_{wake}). In some implementations, the sleep session is defined as the total sleep time (TST). In some implementations, a sleep session is defined as starting at the go-to-sleep time (t_{GTS}) and ending at the wake-up time (t_{wake}). In some implementations, a sleep session is defined as starting at the go-to-sleep time (t_{GTS}) and ending at the rising time (tᵣᵢₛₑ). In some implementations, a sleep session is defined as starting at the enter bed time (t_{bed}) and ending at the wake-up time (t_{wake}). In some implementations, a sleep session is defined as starting at the initial sleep time (tₛₗₑₑₚ) and ending at the rising time (tᵣᵢₛₑ).

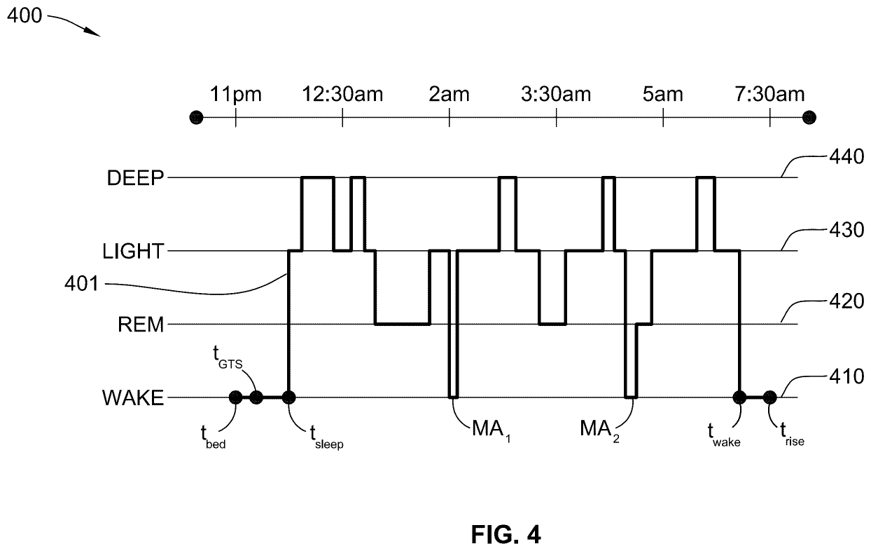

Referring to FIG. 4, an exemplary hypnogram 400 corresponding to the timeline 300 (FIG. 3), according to some implementations, is illustrated. As shown, the hypnogram 400 includes a sleep-wake signal 401, a wakefulness stage axis 410, a REM stage axis 420, a light sleep stage axis 430, and a deep sleep stage axis 440. The intersection between the sleep-wake signal 401 and one of the axes 410-440 is indicative of the sleep stage at any given time during the sleep session.

The sleep-wake signal 401 can be generated based at least in part on physiological data associated with the user (e.g., generated by one or more of the sensors 130 described herein). The sleep-wake signal can be indicative of one or more sleep stages, including wakefulness, relaxed wakefulness, microawakenings, a REM stage, a first non-REM stage, a second non-REM stage, a third non-REM stage, or any combination thereof. In some implementations, one or more of the first non-REM stage, the second non-REM stage, and the third non-REM stage can be grouped together and categorized as a light sleep stage or a deep sleep stage. For example, the light sleep stage can include the first non-REM stage and the deep sleep stage can include the second non-REM stage and the third non-REM stage. While the hypnogram 400 is shown in FIG. 4 as including the light sleep stage axis 430 and the deep sleep stage axis 440, in some implementations, the hypnogram 400 can include an axis for each of the first non-REM stage, the second non-REM stage, and the third non-REM stage. In other implementations, the sleep-wake signal can also be indicative of a respiration signal, a respiration rate, an inspiration amplitude, an expiration amplitude, an inspiration-expiration amplitude ratio, an inspiration-expiration duration ratio, a number of events per hour, a pattern of events, or any combination thereof. Information describing the sleep-wake signal can be stored in the memory device 114.

The hypnogram 400 can be used to determine one or more sleep-related parameters, such as, for example, a sleep onset latency (SOL), wake-after-sleep onset (WASO), a sleep efficiency (SE), a sleep fragmentation index, sleep blocks, or any combination thereof.

The sleep onset latency (SOL) is defined as the time between the go-to-sleep time (t_{GTS}) and the initial sleep time (tₛₗₑₑₚ). In other words, the sleep onset latency is indicative of the time that it took the user to actually fall asleep after initially attempting to fall asleep. In some implementations, the sleep onset latency is defined as a persistent sleep onset latency (PSOL). The persistent sleep onset latency differs from the sleep onset latency in that the persistent sleep onset latency is defined as the duration time between the go-to-sleep time and a predetermined amount of sustained sleep. In some implementations, the predetermined amount of sustained sleep can include, for example, at least 10 minutes of sleep within the second non-REM stage, the third non-REM stage, and/or the REM stage with no more than 2 minutes of wakefulness, the first non-REM stage, and/or movement therebetween. In other words, the persistent sleep onset latency requires up to, for example, 8 minutes of sustained sleep within the second non-REM stage, the third non-REM stage, and/or the REM stage. In other implementations, the predetermined amount of sustained sleep can include at least 10 minutes of sleep within the first non-REM stage, the second non-REM stage, the third non-REM stage, and/or the REM stage subsequent to the initial sleep time. In such implementations, the predetermined amount of sustained sleep can exclude any micro-awakenings (e.g., a ten second micro-awakening does not restart the 10-minute period).

The wake-after-sleep onset (WASO) is associated with the total duration of time that the user is awake between the initial sleep time and the wake-up time. Thus, the wake-after-sleep onset includes short and micro-awakenings during the sleep session (e.g., the micro-awakenings MA₁ and MA₂ shown in FIG. 4), whether conscious or unconscious. In some implementations, the wake-after-sleep onset (WASO) is defined as a persistent wake-after-sleep onset (PWASO) that only includes the total durations of awakenings having a predetermined length (e.g., greater than 10 seconds, greater than 30 seconds, greater than 60 seconds, greater than about 5 minutes, greater than about 10 minutes, etc.)

The sleep efficiency (SE) is determined as a ratio of the total time in bed (TIB) and the total sleep time (TST). For example, if the total time in bed is 8 hours and the total sleep time is 7.5 hours, the sleep efficiency for that sleep session is 93.75%. The sleep efficiency is indicative of the sleep hygiene of the user. For example, if the user enters the bed and spends time engaged in other activities (e.g., watching TV) before sleep, the sleep efficiency will be reduced (e.g., the user is penalized). In some implementations, the sleep efficiency (SE) can be calculated based at least in part on the total time in bed (TIB) and the total time that the user is attempting to sleep. In such implementations, the total time that the user is attempting to sleep is defined as the duration between the go-to-sleep (GTS) time and the rising time described herein. For example, if the total sleep time is 8 hours (e.g., between 11 PM and 7 AM), the go-to-sleep time is 10:45 PM, and the rising time is 7:15 AM, in such implementations, the sleep efficiency parameter is calculated as about 94%.

The fragmentation index is determined based at least in part on the number of awakenings during the sleep session. For example, if the user had two micro-awakenings (e.g., micro-awakening MA₁ and micro-awakening MA₂ shown in FIG. 4), the fragmentation index can be expressed as 2. In some implementations, the fragmentation index is scaled between a predetermined range of integers (e.g., between 0 and 10).

The sleep blocks are associated with a transition between any stage of sleep (e.g., the first non-REM stage, the second non-REM stage, the third non-REM stage, and/or the REM) and the wakefulness stage. The sleep blocks can be calculated at a resolution of, for example, 30 seconds.

In some implementations, the systems and methods described herein can include generating or analyzing a hypnogram including a sleep-wake signal to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof based at least in part on the sleep-wake signal of a hypnogram.

In other implementations, one or more of the sensors 130 can be used to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof, which in turn define the sleep session. For example, the enter bed time t_{bed} can be determined based at least in part on, for example, data generated by the motion sensor 138, the microphone 140, the camera 150, or any combination thereof. The go-to-sleep time can be determined based at least in part on, for example, data from the motion sensor 138 (e.g., data indicative of no movement by the user), data from the camera 150 (e.g., data indicative of no movement by the user and/or that the user has turned off the lights), data from the microphone 140 (e.g., data indicative of the using turning off a TV), data from the user device 170 (e.g., data indicative of the user no longer using the user device 170), data from the pressure sensor 132 and/or the flow rate sensor 134 (e.g., data indicative of the user turning on the respiratory therapy device 122, data indicative of the user donning the user interface 124, etc.), or any combination thereof.

Generally, a user who is prescribed usage of the respiratory therapy system 120 will tend to experience higher quality sleep and less fatigue during the day after using the respiratory therapy system 120 during the sleep compared to not using the respiratory therapy system 120 (especially when the user suffers from sleep apnea or other sleep related disorders). For example, the user 210 may suffer from obstructive sleep apnea and rely on the user interface 124 (e.g., a full face mask) to deliver pressurized air from the respiratory therapy device 122 via conduit 126. The respiratory therapy device 122 can be a continuous positive airway pressure (CPAP) machine used to increase air pressure in the throat of the user 210 to prevent the airway from closing and/or narrowing during sleep. For someone with sleep apnea, her airway can narrow or collapse during sleep, reducing oxygen intake, and forcing her to wake up and/or otherwise disrupt her sleep. The CPAP machine prevents the airway from narrowing or collapsing, thus minimizing the occurrences where she wakes up or is otherwise disturbed due to reduction in oxygen intake. While the respiratory therapy device 122 strives to maintain a medically prescribed air pressure or pressures during sleep, the user can experience sleep discomfort due to the therapy.

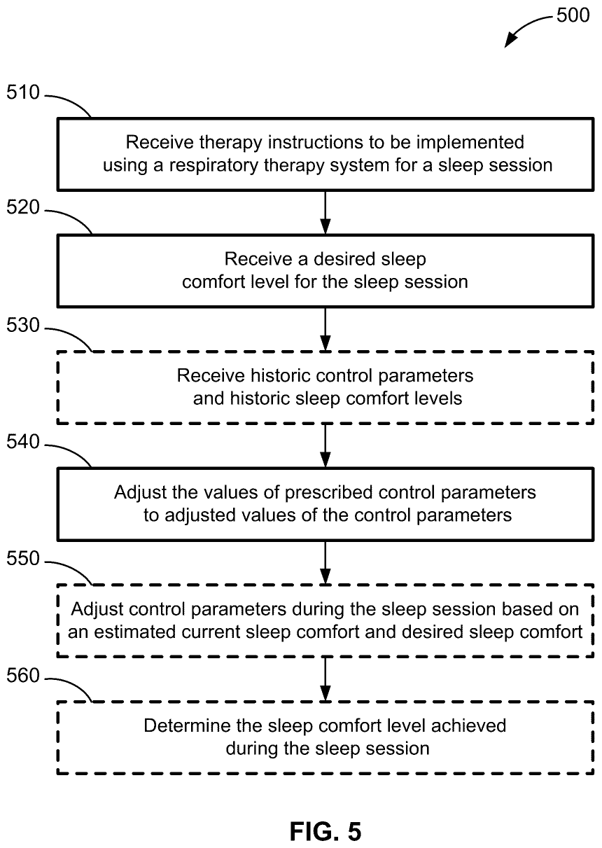

FIG. 5 illustrates a method 500 for optimizing sleep for a user of a respiratory therapy system (such as respiratory therapy system 120) that includes a respiratory therapy device configured to supply pressurized air (such as respiratory therapy device 122), and a user interface (such as user interface 124) coupled to the respiratory therapy device via a conduit (such as conduit 126). The user interface is configured to engage with the user, and aids in directing the pressurized air to the user's airway. Generally, a control system having one or more processors (such as control system 110 of system 100) is configured to carry out the steps of method 500. A memory device (such as memory device 114 of system 100) can be used to store machine-readable instructions that are executed by the control system to carry out the steps of method 500. The memory device can also store any type of data utilized in the steps of method 500. Generally, method 500 can be implemented using a system (such as system 100) that includes the respiratory therapy system, the control system, and the memory device.

At step 510, therapy instructions to be implemented using the respiratory therapy system 120 for a sleep session are received. The therapy instructions can be entered or pre-programed into the respiratory therapy system 120 by a care provider or by the user 210. The therapy instructions can include a plurality of prescribed control parameters. For example, the therapy instructions can include a prescribed pressure and pressure ranges (e.g., a target pressure, a minimum and maximum pressure). The therapy instruction can also include a prescribed ramp rate, or ranges of ramp rates, to achieve the prescribed pressure or pressures.

In some implementations the control parameter includes a sound, an Expiratory Pressure Relief (EPR) setting, a humidification level, a device movement, a light turning on or changing in brightness, a fan turning on or changing in output, or any combination thereof.

Sounds that can be used as a control parameter include, without limitation, white noise, pink noise, brown noise, violet noise a soothing sound, music, an alarm, an alert, beeping, or a combination thereof. As used herein, some variants of the flat shaped white noise sound are referred to as pink noise, brown noise, violet noise, etc. In some implementations the sounds (e.g., white noise, pink noise, brown noise, violet, etc.) aid in masking noises from the respiratory therapy system or the environment. In some implementations, the sounds (e.g., soothing sounds and music) aid the user or bed partner to remain in a sleep state or can gently awaken or cause the user to change sleeping position, e.g., to a position to maintain a sleep session or sleep state.

In some implementations the sound can be provided by the one or more speakers 142 of system 100. Optionally, the system 100 includes multiple speakers 142 to provide localized sound emission. The speakers 142 can include in the car speakers, over the car speakers, adjacent to the ear speakers, ear buds, ear pods, or any combination thereof. The speakers 142 can be wired or wireless speakers (e.g., headphones, bookshelf speakers, floor standing speakers, television speakers, in-wall speakers, in-ceiling speakers, etc.). In some implementations, the speakers 142 are worn by the user 210 and/or the bed partner 220. In some such implementations, the provided speakers 142 can supply a masking noise without impacting the bed partner as the sound would be localized via the type of the speakers 142. In such implementations, respective localized speakers 142 could be provided for the respiration user 210 and/or the bed partner 220.

Optionally, the speaker 142 is attached to one or more of a strap or strap segments of the user interface 124. Thus, the user 210 (FIG. 2) and/or the bed partner 220 has the choice to perceive a relatively flat shaped white noise sound, or for a quieter (lower level and/or low pass filtered) shaped noise signal. In some such implementations, the higher frequency sounds/noises (e.g., “harsher” sounds) are reduced, while still providing masking sounds to the environmental noise. The system 100 can select an optimized set of fill-in sound frequencies to achieve a target noise profile. For example, if certain components of sound already exist in the frequency spectrum (e.g., related to a box fan in the room, a CPAP blower motor, etc.), then the system 100 can select fill-in sounds with sound parameters/characteristics that fill in the quieter frequency bands, for example, up to a target amplitude level. Thus, the system 100 is able to adaptively attenuate the higher and/or lower frequency components using active adaptive masking and/or as adaptive noise canceling such that the perceived sound is more pleasant and relaxing to the ear (the latter being more suited to more slowly varying and predictable sounds).

In general, EPR is a feature on some respiratory therapy devices (e.g., CPAP machines) that allows users to adjust between different comfort settings to alleviate feelings of breathlessness some users experience. For example, a drop of 2 cm H₂O between inspiration and expiration. Where this feature can be manual, the EPR setting can be implemented by the system 100 without direct manual user input according to some implementations of this description to provide a desired sleep comfort.

In some implementations a control parameter is the humidity of the air delivered to the user 210. For example, the humidity level can be chosen to mitigate discomfort due to dryness of the sinuses or mouth. The humidity level can also be chosen to optimize a seal between the interface 124 and user 210.

A device that is actuated or moved as a control parameter can include, without limitation, a smart pillow, an adjustable bed frame, an adjustable mattress, a fan, an adjustable blanket, or any combination thereof. For example, the device is under control of control system 110. In some implementations the smart pillow, smart mattress, or adjustable blanket can include one or more inflatable compartments or bladders that can inflate or deflate. The actuated device can thereby change the orientation of user. For example, a pillow 260 can be a smart pillow including one or more inflatable bladders that change the orientation of user 210 if the user's head is in an orientation that increases the likelihood of increasing the AHI. In another or additional implementation, an adjustable bed frame can include sections that can rise or lower as driven by a motor and cause the user 210 to change orientation, e.g., forcing user 210 to roll from their side to their back. In some implementations, the fan, for example a fan placed on nightstand 240, a fan in a window or a ceiling fan, turns on responsive to the likelihood of an AHI increasing due to orientation of the user 210. In some implementations, the fan generates white noise. The fan can increase in speed and movement of air gradually, so as to not wake and/or disturb the user 210 and/or the bed partner 220 with a sudden change of air movement or sound from the fan.

In some implementations the control parameter is injection of a substance into the pressurized air to be being delivered to the user interface 124. For example, receptacle 182 can be charged with a substance, the receptacle having an outlet that is in direct or indirect fluid communication with the conduit 126. The substance can be configured or selected to invoke a physical reaction by the user 210. For example, the user 210 may change orientation.

Optionally, the substance can include a medicament, such as anti-inflammatory medicine, medicine to treat an asthma attack, medicine to treat a heart attack, etc. Generally, any type of medicament that is used to treat any ailment, symptom, disease, etc. can be delivered to the airway of the user 210. When the substance is a medicament, the substance generally includes one or more active ingredients, and one or more excipients. The excipients serve as the medium for conveying the active ingredient, and can include substances such as bulking agents, fillers, diluents, antiadherents, binders, coatings, colors, disintegrants, flavors, glidants, lubricants, preservatives, sorbents, sweeteners, vehicles, or any combinations thereof. The active ingredient is generally the portion of the medicament that actually causes the effect brought on by the medicament.

The substance can also optionally be an aroma compound (e.g., a substance that delivers scents and/or aromas to the airway of the user 210), a sleep-aid (e.g., a substance that aids the user 210 in falling asleep), a consciousness-arousing compound (e.g., a substance that aids the user 210 in waking up, also referred to as a sleep inhibitor), a cannabidiol oil, an essential oil (such as lavender, valerian, clary sage, sweet marjoram, roman chamomile, bergamot, etc.). The substance can generally be a solid, a liquid, a gas, or any combination thereof. The substance can alternatively or additionally include one or more nanoparticles.

In some implementations, the prescribed control parameters for a therapy is a function of the sleep stage or sleep architecture. For example, according to the user's prescription, in transitioning from an awake state to an N1 stage, a pressure ramp may be initiated to achieve a first target pressure from ambient pressure. The ramp can be implemented so as to gradually accustom the user 210 to the pressure change. During a following N2 and N3 the first target pressure may be maintained according to the user's prescription. Since during REM most users are more likely to experience an increase in apneas, a higher pressure from a previous sleep state is often prescribed for REM sleep stages. Accordingly, a ramp to the higher pressure can be implemented for REM sleep. The sleep state can be determined, as previously described, by monitoring physiological parameters using sensors (e.g., one or more sensors 130, blood pressure device 180, or activity tracker 190 of FIG. 1).

At step 520, a desired sleep comfort level is entered into the respiratory therapy system 120. The desired sleep comfort may be selected based on how important the sleep comfort is to the user 210 for the sleep session. For example, a first time user 210 might be encouraged to select a high comfort level. A more experienced user 210 may not prioritize sleep comfort or they do not feel any significant discomfort when using the respiratory therapy system 120 with the prescribed control settings. In some instances, a user 210 may generally desire a high sleep comfort but has need for high quality sleep and is willing to have a lower comfort level for a specific sleep session and so selects a low desired sleep comfort level.

Alternatively or in addition to the user setting the desired sleep comfort level, in some implementations, the system 100 can automatically set a sleep comfort level for the user 210. The automatically set sleep comfort level can be based at least in part on data associated with the user 210 and or data based on the user's experience and/or length of time and/or hours using the respiratory therapy system 120 and/or sleep therapy. For example, the automatically set sleep comfort level can be set based on the number of days the user has been using sleep therapy, or the number of logged hours using a respiratory therapy system 120.

In some implementation, one aspect of sleep comfort relates to how the user 210 or a population of users rate a sleeping experience. The user or users can, after a sleeping session, rate the sleep comfort experience. The rating system can include different criteria including, for example, data of reported symptoms of aerophagia, difficulty in getting to sleep, dry sinuses/mouth, muscle soreness, and dry skin. The criteria can be subdivided and quantified such as by rating any pain due to aerophagia from low (e.g., mild gas), medium lingering discomfort (e.g., bloating), to high (e.g., cramps). Difficulty in getting to sleep can be rated as how many times the user might recall checking the time or noting noise or air pressure from the respiratory therapy system 120. Incidences of dry skin, sinuses or mouth, and muscle soreness can also be reported and used to rate the sleep comfort. The rating can be facilitated by a questionnaire with or without care provider's help. In some implementations, the questionnaire may also gather information such as whether the user 210 experiences restless sleep, insomnia, arousals during pressure therapy, bed partner's sleep comfort ratings (e.g., how it affects the bed partner) and other motivational feedback questions, such as increased activity in the following day after pressure therapy, etc. The questionnaire can be presented and the ratings entered by an interactive app via the user interface 124, a touchscreen of the respiratory device 122, a voice input, or an external device 170 (e.g., a smart phone).

An example of a short questionnaire for a user (Joe) rated sleep comfort is shown in Table 1.

TABLE 1
User Reported Sleep Comfort Data
User: Joe
Date: Sep. 1, 2020

| | Sleep Comfort | | | | |
|---|---|---|---|---|---|
| | Low---------------------------------------------High | | | | |
| Number rating → | 1 | 2 | 3 | 4 | 5 |
| Aerophagia | | x | | | |
| Dry sinuses/mouth | | | x | | |
| Dry skin (e.g., eye) | | | x | | |
| Going to sleep | x | | | | |
| Muscle soreness | | x | | | |
| Sleep quality | | | | x | |

The example questionnaire rates the sleep comfort (or discomfort) due to aerophagia, dry sinuses/mouth, dry skin, while going to sleep, muscle soreness, and sleep quality. The questionnaire rates these factors from low to high with 5 possible increments. Sleep quality relates to how well rested user 210 feels. For example, being clear headed and alert. Where the sleep quality does not characterize sleep comfort, it can often inversely relate to the sleep comfort. Including sleep quality in the sleep comfort rating can help in determining how much the sleep comfort can be modified without reducing the sleep comfort to levels where the therapy is not effective.

A total rated sleep comfort score can be determined as a function of the sleep comfort for each item listed in Table 1. Equation 1 shows one implementation of how a sleep comfort rating questionnaire can be used to provide a total rated sleep score.

$$Total Rated Sleep Comfort Score = ( {m}_{1} ) ( aerophagia ) + ( {m}_{2} ) ( dry sinuses / mouth ) + ( {m}_{3} ) ( dry skin ) + ( {m}_{4} ) ( going to sleep ) + ( {m}_{5} ) ( muscle soreness ) - ( {m}_{6} ) ( sleep quality ) ; \qquad Equation 1$$

In Equation 1, m₁ is a weighting factor selected for aerophagia, m₂ is a weight factor selected for dry sinuses/mouth, m₃ is a weight factor for dry skin, m₄ is a weight factor for going to sleep, m₅ is the weight factor for muscle soreness, and m₆ is a weight factor for sleep quality. The Total Rated Sleep Comfort Score value can also be normalized e.g., to have a minimum of 0 or 1, and maximum of 5, 10, 20, 50 or 100. The weight factor relates to the importance of a specific item to sleep comfort. For example, the “aerophagia” item may ultimately be more important to sleep quality than the “going to sleep” item and a greater weight factor is selected or assigned for aerophagia than for going to sleep. The weight factors can be assigned by a first machine learning algorithm, for example, by providing data from multiple sleep sessions for one or more users. A “true” sleep comfort level for each individual sleep session is also used for training the first algorithm. Here the “true” sleep comfort refers to a user, or users, provided overall assessment of the sleep comfort, which can be assigned a value for training the first machine learning algorithm.

The first machine learning algorithm can also determine the overall function, including items other than those listed in Table 1, to provide the most accurate total rated sleep comfort score. In some implementation, the first machine learning algorithm can learn how an individual user 210 rates criteria that is subjective. For example, a first user may rate a sore muscle as more detrimental to sleep comfort than a second user. The first algorithm can learn this difference between the first and second user and accordingly modify the function to determine the Total Rated Sleep Comfort Score, depending on which user is using the respiratory therapy system 120. For example, if equation 1 is used, the weighing factor m₅ associated with muscle soreness for the first user would be less than the same weighing factor for the second user.

In some implementations, an aspect of sleep comfort relates to monitoring a user 210 or users of the respiratory therapy system 120 with sensors, such as sensors 130, during sleep sessions. In some implementations, the sleep architecture for the sleep session is determined as previously described, and the achieved sleep comfort level is determined as a function of sleep architecture. For example, a user 210 may be detected as moving during a non-REM sleep session. A user may be unconsciously trying to, or succeeding at, remove a user interface 124 in an N1 or other sleep phase. In some implementations, a user may be in a sleep position in a non-REM sleep phase (e.g., back vs side) which leads to poor sleep comfort. These activities can be factors influencing sleep comfort and can be monitored using sensors such as motion sensor 138, camera 150, or microphone 140, blood pressure device 180, activity tracker 190, or any combination thereof. Other indicators of sleep comfort that can be monitored can include noises from the respiratory therapy system 120 such as a pump or a leak at the user interface 124 (e.g., a mask leak). The ambient temperature as measured by temperature sensor 136 can also be indicative of sleep comfort. For example, a temperature that is higher or lower than an ideal temperature (e.g., 63° F.) can impact sleep comfort. While a user 210 may not instantly know how the various sensor monitored factors are impacting their sleep, the sensors can monitor these in real time as well as track and provide the data for analyses after the sleep session.

An example of some factors that can be monitored using sensors is shown by Table 2.

TABLE 2
Measured Sleep Comfort factors
User: Joe
Date: Sep. 1, 2020

| | Value |
|---|---|
| # non-REM | 50 |
| movements | |
| Percent time on | 40% |
| back vs side | |
| Average | 65° F. |
| temperature of | |
| room | |
| # of mask leaks | 5 |
| # incidences of | 2 |
| noises >40 dB | |
| AHI | 10 |

Table 2 lists the number of non-REM movements, percent time on back vs side, average room temperature, number of mask leaks, number of incidence of noises above a whisper (e.g., about 40 dB), and AHI. Where AHI is not a direct measure of sleep comfort it can be inversely proportional or otherwise counter to the sleep comfort. Including AHI can provide a balancing consideration since reducing AHI is an important objective of sleep therapies for various sleeping disorders.

A total measured sleep comfort score can be determined as a function of these sensor measurable factors. Any useful function can be implemented. An embodiment of a simple function is shown by equation 2.

$$Total Measured Sleep Comfort Score = ( {m}_{7} ) ( N - R E M Movements ) + ( {m}_{8} ) ( \% Time on Back / Side ) + ( {m}_{9} ) ( Ave . Rm . Temp ) + ( {m}_{10} ) ( \# of Mask Leaks ) + ( {m}_{11} ) ( \# Noises ) - ( {m}_{12} ) ( A H I ) ; \qquad Equation 2$$

where m₇ is a weighting factor selected for N-REM movements, m₈ is a weight factor selected for % time on back/side, m₉ is a weight factor for average room temperature, m₁₀ is a weight factor for number of mask leaks, m₁₁ is a weight factor for number of noise incidences >40 dB, and m₁₂ is a weight factor for AHI. The weight factors, and the overall form of the function, can be determined by using a second machine learning algorithm. The Total Measured Sleep Comfort Score value can also be normalized e.g., to have a minimum of 0 or 1, and maximum of 5, 10, 20, 50 or 100. Data from the user or multiple users over multiple sleep sessions can be input into the second machine learning algorithm. A true sleep comfort can be used for training the second machine learning algorithm. In some implementations, the first machine learning algorithm provides a rated sleep comfort which can be used to train the second machine learning algorithm to determine the measured sleep comfort. For example, the rated sleep comfort from the first algorithm is used as the true sleep comfort for training the second algorithm.

In some implementations data from user 210 rated sleep comfort (e.g., Table 1) is combined with data from sensor measured sleep comfort factors (e.g., Table 2). For example, an overall sleep comfort score can be determined using user reported sleep comfort and measured sleep comfort. For example, a function for an Overall Sleep Comfort Score can be a combination of equation 1 and 2. The Overall Sleep Comfort Score value can also be normalized e.g., to have a minimum of 0 or 1, and maximum of 5, 10, 20, 50 or 100. In some implementations the first algorithm and second algorithm are combined as a single machine learning algorithm.

Returning to FIG. 5 and step 520, the desired sleep comfort level can be a value selected from a series of incrementally increasing values between a first value, indicative that the comfort experience is not important to the user, and a second value, indicative that the user desires the best possible sleep comfort experience. The values can be scaled similarly to the sleep comfort score that is used i.e., the Rated Sleep Comfort Score (e.g., Table 1, equation 1), the Measured Sleep Comfort Score (e.g., Table 2, equation 2) or the Overall Sleep Comfort Score (e.g., the combination of the Rated Sleep Comfort Score and the Measured Sleep Comfort Score). For example, the minimum and maximum values for the various scores correspond to the minimum and maximum values the user 210 can select for the desired sleep comfort. The values can be on a continuous scale, such as an analog volume control, or they can be digital. In other implementations, the sleep comfort can have digital value. For example, the values can be integers between 1 and 10, where 1 is indicative that the user does not desire an improvement in sleep comfort, and where 10 indicates the user desires the best possible sleep comfort experience. The sleep comfort level can be selected or dialed in according to the desired sleep comfort of the user.

While in some implementations the desired comfort level is selected when a user 210 goes to bed for a sleep session, in some other implementation the comfort level can be changed by the user 210 during the sleep session. For example, a user 210 may wake up after some combination of non-REM and REM sleep and decide that they are uncomfortable or can't get back to sleep. The user 210 can accordingly decide to increase the sleep comfort level. Alternatively, the user 210 may wake up during a sleep session and notice the time is 4 am and decide they need to get a couple more hours of high quality sleep so they decrease the sleep comfort to improve their sleep quality.

As shown by step 530, in some optional implementations, historic control parameters and historic sleep comfort levels can be entered into the respiratory therapy system 120. As used here, “historic” relates to one or more previous sleep sessions. For example, a historic sleep comfort might be a user selected value of 8 (e.g., on a scale of 1-10) which the user may have selected for a sleep session just prior to the current sleep session. In this instance, the historic control parameters are control parameters from the previous sleep session implemented using the respiratory therapy system 120 to target the desired sleep comfort of 8. A user may, after assessing the previous sleep session, determine that the comfort level actually achieved (the historic sleep comfort) is lower or higher than what they had entered. The received historic parameters and sleep comfort can be used for the current sleep session to more accurately achieve the desired sleep comfort for the sleep session where the user input indicates a gap between. For example, the user can select a higher or lower sleep comfort level based on their personal experience to self-titrate the desired sleep comfort.

In some implementations, the use of historic data relates to training of the system and can be implemented with the use of artificial intelligence. For example, a third machine learning algorithm can include the data used in the first machine learning algorithm (user reported sleep comfort), the second machine training algorithm (user measured sleep comfort), and a historic adjusted control parameter. In some implementations, the third machine learning algorithm is a combination of or includes elements from the first and second machine training algorithms.

In step 540, the control parameters are adjusted from the prescribed control parameters and the adjusted control parameters are implemented during the sleep session. Where the prescribed control parameters are implemented to improve the sleep quality of the user 210, the adjusted control parameters are implemented to achieve the desired sleep comfort level of the user 210.

In some implementations, the received therapy instructions are provided to aid a user in achieving a target therapy parameter during a sleep session, and the adjusted one of more values or range of values of the plurality of control parameters provides a therapy parameter that is different from the target therapy parameter. In some implementations, the adjusted one or more of the values or the range of values of the plurality of control parameters provides a therapy parameter that is greater than the target therapy parameter. In some other implementations, the adjusted one or more of the values or the range of values of the plurality of control parameters provides a therapy parameter that is less than the target therapy parameter.

In some implementations the received therapy instructions are provided to aid a user in achieving a target AHI for the user during the sleep session. While in some implementations, the achieved AHI is about the same as the target for the therapy, in some other implementation the adjusted control parameter can lead to an AHI that is greater than (e.g., worse than) the target AHI.

The sleep comfort can therefore be improved at the expense of a degradation in the quality of the sleep. In some implementations, the control parameters are adjusted to maximized the sleep quality and maximize the sleep comfort. For example, maximizing sleep quality and sleep comfort can be a feature of a machine learning algorithm such as the third machine learning algorithm.

In some implementations the pressure, range of pressures, or pressure ramps are adjusted up or down from the prescribe pressure, ranges of pressures, or pressure ramps. In some implementations, the pressure, range of pressures, or pressure ramps are adjusted to be lower than the prescribed pressure for at least a portion of the sleep session. In some implementations, the pressure, range of pressures, or pressure ramps are adjusted to be higher than the prescribed pressure for at least a portion of the sleep session. In some implementations, the pressure or range of pressures are adjusted to be higher than the prescribed pressure for at least a portion of the sleep session, and the average adjusted pressure during the entire sleep session is lower than the average prescribed pressure for the entire sleep session.

In some implementations, sounds that are provided by the one or more speakers 142 of system 100 are adjusted from prescribed sounds. In some embodiments, white noise, pink noise, brown noise, violet noise a soothing sound, or music is increased in volume, decreased in volume, increased in duration or decreased in duration from the prescription. In some implementations an alarm or alert that indicates a poor sleeping position with respect to sleep quality is turned off. The silencing of the alarm allows the user 210 to continue sleeping, albeit in a poor position, but providing more sleep comfort.

In some implementations, an EPR setting is adjusted up or down from a prescribed value. For example, where an EPR setting is a drop of 0.5 cm H₂O between inspiration and expiration as prescribed, the adjusted setting can be 1 cm H₂O, 1.5 cm H₂O, or 2.0 cm H₂O.

In some implementations, the humidity of the air delivered to the user 210 is adjusted up or down from a prescribed value. For example, in some implementations the humidity is increased to mitigate discomfort due to dry skin or dry sinuses/mouth. In some other implementations the humidity is decreased to mitigate discomfort due to the user 210 feeling discomfort due to slickness or stickiness of the user interface 124 (e.g., a face mask). The decrease in humidity can lead to a decrease in sleep quality, for example, due to increase face leaks, but the sleep comfort is increased.

In some implementations an adjusted control parameter includes a device that is actuated or moved. In a prescribed therapy the device may cause a user 210 to change position to reduce or avoid a mask leak. Where a user 210 may tend to move to a position that causes a mask leak, the repeated actuation of the device to try and force the user to a different position might cause sleep discomfort. For, example, forcing the user to assume a position that causes muscle soreness or causes acrophagia.

In implementations where the control parameter is injection of a substance into the pressurized air to be being delivered to the user interface 124, the adjustment can be a decrease or increase in the delivered substance. For example, a consciousness-arousing compound can be prescribed to limit the sleep session. As adjusted, the delivery of the substance can be delayed to a later time in the sleep session thereby prolonging the sleep session and improving the sleep quality.

In some implementations the prescribed control parameters maintain an ideal sleep architecture. The adjusted control parameters can change the ideal sleep architecture. For example, with adjusted control parameters less REM might be occur due to an increase in apneas occurring.

In some implementations, the selected desired sleep comfort level does not provide any measurable increase in sleep quality but can be used to allow a user 210, such as a first time user, to adopt the sleep therapy. The user 210 can gradually, optionally with guidance from a care provider, decrease the comfort level to improve the sleep quality in a “weaning” process. In some implementations, the weaning process is part of a program extending over several days, weeks or months and can be an automatically implemented feature of the control system 110. In some implementations, the weaning program can be a feature of one or more of the machine learning algorithms described herein.

Step 550 shows an optional implementation wherein the control parameters are adjusted during the sleep session responsive to a current sleep comfort and the desired sleep comfort. The current sleep comfort is an estimated sleep comfort of the user and does not require any direct or conscious input from the user. For example, the current sleep comfort can be determined by monitoring the user 210 using sensors such as sensors 130, blood pressure device 180, or activity tracker 190 of FIG. 1. Where Table 2 shows measured sleep comfort for an entire sleep session, the various factors that can be monitored using sensors can be sampled and tallied during a sleep session. How these factors change during the sleep session can be used to predict the sleep comfort that will be achieved during the sleep session. Where the trajectory of the predicted sleep comfort based on the current sleep comfort deviates from the desired sleep comfort level, corrective action can be implemented. The corrective action can be implemented by modification of the control parameters. For example, if the temperature is high and predicted to decrease the sleep comfort, a thermostat can be reset or a fan turned on. If a muscle soreness is predicted due to a user 210 sleeping position, a device such as a smart pillow, smart mattress, or adjustable blanket can be activated to cause the user 210 to change position. The prediction can be implemented using a prediction algorithm. The prediction algorithm can be a fourth machine learning algorithm, that can include the previously described first, second and third algorithms.

Step 560 is an optional step that includes determining the sleep comfort level achieved by user 210 in the sleep session. The sleep comfort level achieved can be determined as previously described, for example, using the user rated sleep comfort and measured sleep comfort. Optionally, the sleep comfort is determined using a fifth machine learning algorithm that can be any combination of the first, second, third and fourth machine learning algorithms previously discussed. In some embodiments the sleep comfort is reported out to the user, for example through external device 170.

According to some implementations, any of the plurality of prescribed control parameters can be adjusted to improve the sleep comfort. For example, the plurality of control parameters can include a prescribed pressure, a range of prescribed pressures, a range of prescribed pressures ramps, and a range of prescribed step pressures changes, and one or more of the prescribed pressure, the range of prescribed pressures, the range of prescribed pressures ramps, and the range of prescribed step pressures changes are adjusted to improve the sleep comfort. In some implementations, the prescribed pressure is adjusted to an adjusted pressure that is less than the prescribed pressure or range of prescribed pressures. In some implementations, the prescribed pressure range is adjusted to a range of pressures that is lower than the prescribed range of pressures. For example, the average or mean of the prescribed pressure range can be adjusted to be lower, or one or more of the maximum or minimum pressure can be adjusted lower.

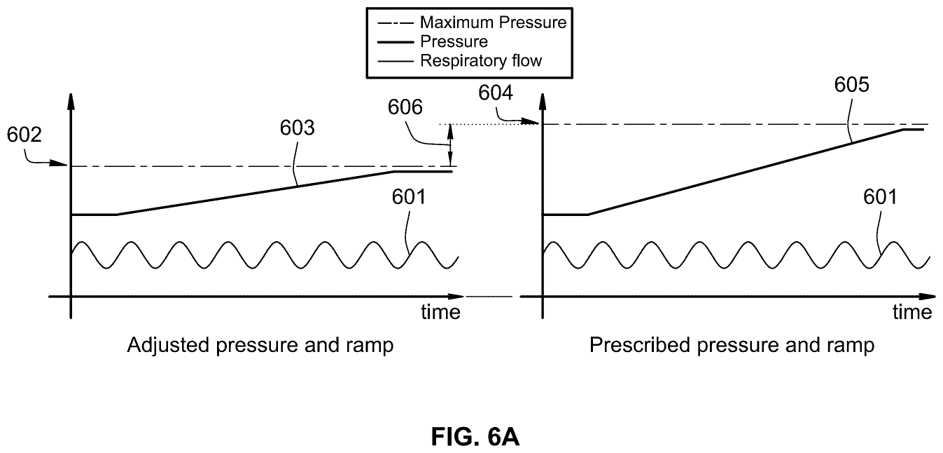

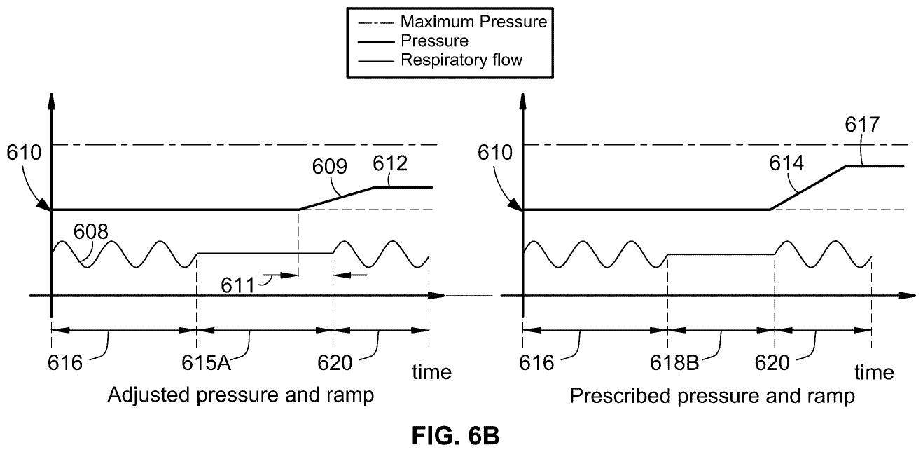

FIG. 6A are plots showing an implementation according to some aspects of the disclosure. The plots in FIG. 6A show pressure ramps that can be implemented at or near the beginning of a sleep session, for example to aiding the user in falling asleep at the beginning of the sleep session. The left side plot shows an adjusted target maximum pressure 602, an adjusted pressure ramp 603, and a respiratory flow 601. The right side plot shows the therapeutic prescribed maximum pressure 604, the prescribed pressure ramp 605, and the respiratory flow 601. A user may be prescribed the prescribed maximum pressure 604 to be implemented with respiratory therapy system 120. The prescribed maximum pressure 604 can be, for example, 15 mm H₂O. This pressure is prescribed to provide a target AHI, such as 10 or less per sleep session. The prescribed target AHI and pressure can be determined, for example, during a titration experiment supervised by a care provider. The user 210 may find the prescribed maximum pressure 604 and/or the prescribed pressure ramp 605 reduces their sleep comfort. For example, the user 210 may have symptoms of acrophagia after a sleep session where the prescribed maximum pressure 604 is implemented. Alternatively, or additionally, the user 210 may find the prescribed pressure ramp 605 increases the pressure too rapidly, making it difficult to fall asleep. The adjusted target maximum pressure 602 and the adjusted pressure ramp 603 are responsive to the user 210 selecting a desired sleep comfort level. For example, a user feeling bloated when the prescribed pressure ramp 605 is implemented, selects a sleep comfort level to decrease the bloating and increase sleep comfort. Likewise, the more gradual increase in the adjusted pressure ramp 603 as compared to the prescribed pressure ramp 605 can provide a gentler transition for user 210 to go to sleep. While the adjusted target maximum pressure 602 and the adjusted pressure ramp 603 can provide better sleep comfort as compared to the prescribed maximum pressure 604 and the prescribed pressure ramp 605, the sleep quality can be reduced. For example, the AHI achieved can be higher using prescribed maximum pressure 604 as compared to the AHI achieved using adjusted target maximum pressure 602.

As another example, the user 210 may find that the prescribed pressure ramp 605 starts at a pressure that is too low, which does not deliver a large enough quantity of breathing air and creates an uncomfortable feeling of hungering for air, called “air hunger”. This may also reduce sleep comfort and make it difficult to fall asleep. The prescribed pressure ramp 605 may then be adjusted by the user to a desired sleep comfort level such that there is an appropriate adjusted pressure ramp 603, which enables the user 210 to provide sufficient breathing air and go to sleep, while on therapy.

The plots illustrated in FIG. 6A also show another aspect according to some implementations. A delta 606 between the prescribed maximum pressure 604 and the adjusted target maximum pressure 602 is shown. Where a higher pressure causes more sleep discomfort, a larger delta 606 indicates the user 120 has selected a higher desired sleep comfort. In comparison, a smaller delta 606 would indicate a selected a lower sleep comfort. Although shown as applied to pressure, other control factors can be similarly manipulated and the delta between a prescribed and adjusted value is responsive to the desired sleep comfort level. In some implementation, an increase in a control parameter will provide better sleep comfort. For example, if the prescribed control parameter is a concentration of a medicament added via receptacle 180, an increase of the medicament, while possibly improving sleep comfort, could cause more apneas. In this instance, the increase in the medicament is an adjustment of a control parameter to a higher level from the prescribed control parameter.

FIG. 6B shows an implementation according to another aspect of the description. The left side plot shows an adjusted pressure and ramp profile. The plots in FIG. 6B show pressure ramps that can be implemented in response to a user experiencing an event during a sleep session. The left side plot shows a respiratory flow 608 and time segments 616, 618A and 620. After time segment 616, an apnea is shown in time segment 618A. In response to the apnea, a ramp 609 from an initial pressure 610 to a second higher pressure 612 is implemented by respiratory therapy system 120. After a delay 611, the apnea is stopped and regular breathing continues in time segment 620. The right side plot shows a prescribed pressure and ramp profile. After time segment 616 at the initial pressure 610, an apnea occurs in time segment 618B. In response, a prescribed pressure ramp 614 to a higher target pressure of 617 is implemented. The prescribed pressure ramp 614 is steeper (positive) than ramp 609, and the pressure 617 is also higher than the second pressure 612. Because of the more aggressive control parameters used in the prescribed therapy, the apnea segment 618B is shorter than the apnea segment 618A. The segment 618B is shorter than 618A according to this implementation because the apnea is more quickly stopped after implementation of the prescribed pressure ramp 614, than after implementation of pressure ramp 609. Specifically, no delay 611, shown in the left side plot, is seen in the right side plot. Although the more aggressive control parameters implemented using the prescribed pressure ramp 614 and pressure 617 can more effectively eliminate an apnea, this can lead to sleep discomfort. The less aggressive pressure ramp 609 and the second pressure 612 reduces the discomfort.

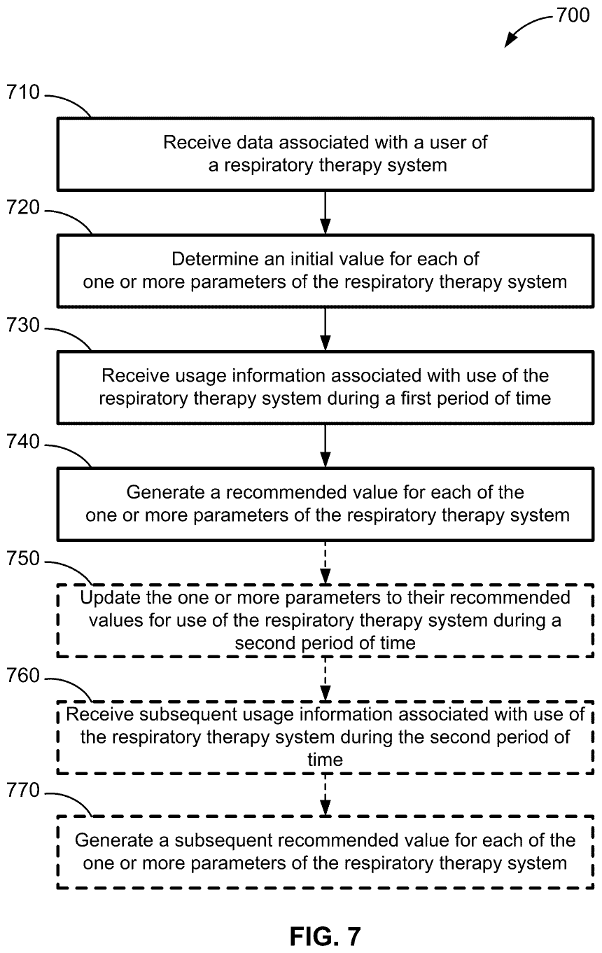

FIG. 7 illustrates a method 700 for optimizing one or more parameters of a respiratory therapy system (such as respiratory therapy system 120). In some implementations, the respiratory therapy system includes a respiratory therapy device configured to supply pressurized air (such as respiratory therapy device 122), and a user interface (such as user interface 124) coupled to the respiratory therapy device via a conduit (such as conduit 126). The user interface is configured to engage with the user, and aids in directing the pressurized air to the user's airway. Generally, a control system having one or more processors (such as control system 110 of system 100) is configured to carry out the steps of method 700. A memory device (such as memory device 114 of system 100) can be used to store machine-readable instructions that are executed by the control system to carry out the steps of method 700. The memory device can also store any type of data utilized in the steps of method 700. Generally, method 700 can be implemented using a system (such as system 100) that includes the respiratory therapy system, the control system, and the memory device.

Generally, the respiratory therapy system is used according to a variety of different parameters. These parameters can include settings of the respiratory therapy system, but may also include other parameters relevant to the user's use of the respiratory therapy system, such as light levels in the room where the user uses the respiratory therapy system, sound levels in the room where the user uses the respiratory therapy system, the position in which the user is in during their use of the respiratory therapy system, and other parameters. All of these parameters contribute to the user's experience when using the respiratory therapy system, and can generally have a number of different values. The values of all of these parameters related to how effective the respiratory therapy system is in treating any issues that the user is currently experiencing (such as SDB and/or OSA).

However, the values of the parameters also impact the user's comfort when using the respiratory therapy system, which in turn can impact the user's compliance with a prescribed use of the respiratory therapy system. Generally, the user's compliance can be measured in a variety of different ways. In some cases, compliance is defined as adherence to the prescribed use of the respiratory therapy system during an initial 90-day period. In other cases, compliance can be defined as short-term adherence or long-term adherence, both of which can depend on external requirements set by insurance guidelines, medical guidelines, industry guidelines (e.g., standards set by industry), government guidelines (e.g., guidelines from the appropriate governing body or regulatory agency), and others. In some cases, compliance is defined as using the respiratory therapy system for a minimum number of days (or sleep sessions) Dₘᵢₙ within a time period containing a total number of days (or sleep sessions) Dₜₒₜ. The minimum number of days Dₘᵢₙ could be defined as a raw number of days, or as a percentage of the total number of days Dₜₒₜ. In some cases, compliance is defined as using the respiratory therapy system for at least a minimum number of hours hₘᵢₙ per use (e.g., per sleep session). The minimum number of hours hₘᵢₙ could be defined as a raw number of hours, or as a percentage of the length of the sleep session. In some cases, compliance can be defined as using the respiratory therapy system for both (i) the minimum number of days Dₘᵢₙ within the total number of days Dₜₒₜ, and (ii) the minimum number of hours hₘᵢₙ per use. For example, in some implementations compliance is defined as using the respiratory therapy system for at least 70% of the first 90 days, and for using the respiratory therapy system for at least 4 hours per night (either across the number of nights the respiratory therapy system was used, or across all 90 nights). As used herein, the term compliance generally refers to adhering to any type of prescribed use or plan of use for a respiratory therapy system, regardless of the length of the prescribed use, the source of the prescribed use, and any factors impacting the prescribed use.

Method 700 is directed to techniques for optimizing a plurality of parameters of the respiratory therapy system, in order to improve the user's comfort when using the respiratory therapy system and in turn improve the user's compliance with the prescribed use of the respiratory therapy system. In many cases, various parameters of the respiratory therapy system can have a range of values while still providing an effective therapeutic benefit to the user. Optimizing the plurality of parameters can include identifying certain values or sub-ranges of values within the broader ranges of values that still provide the therapeutic benefit to the user, but improve the user's comfort while using the respiratory therapy system.

In step 710, data associated with a user of the respiratory therapy system (also referred to herein as user data) is received. In some implementations, the received data is data that is specific to the user, e.g., personal data and/or demographic data. The received data can include the user's age, sex, gender, and other physical characteristics of the user. The received data can also include clinical data of the user, which generally associated with the user's use of the respiratory therapy system. For example, the clinical data can include data related to the user's AHI associated with prior use of the respiratory therapy system, prescribed operating parameters of the respiratory therapy system (e.g., prescribed maximum pressure of the pressurize air, prescribed pressure ramp parameters, etc.), the user's sleepiness/restlessness score or scores, the user's reasons for using the respiratory therapy system (e.g., data associated with SDB and/or OSA experienced by the user), or any other relevant clinical and/or medical data. The received data can also indicate what type of user interface that the user wears when using the respiratory therapy system (e.g., face mask, nasal pillows, etc.), what type of sleep tests the user has taken, and other factors. A sleep test (sometimes referred to as a sleep study or polysomnography) is performed by monitoring the user using various sensors during a sleep session. These sensors can include an EEG sensor, an EOG sensor, an EMG sensor, an ECG sensor, and other sensors. The sleep study can be used to determine whether a user experiences OSA or SDB during the sleep session, as well as whether the user is suffering from any other conditions. Sleep tests can be home-based (e.g., performed at home by the user in their own bed), laboratory-based (e.g., performed while the user is in a clinical setting, such as a healthcare facility), or can be performed in other environments.

The received data can also be associated with the user's preferences for use of the respiratory therapy system. These preferences could be related to the type of user interface the user prefers to wear, the position the user prefers to sleep in, the light and/or sound levels in the room during the sleep session, how the respiratory therapy system adjusts the pressure of the pressurized air to deal with events (such as apneas), the pressure of the pressurized air as the user is attempting to fall asleep, balances between lower pressures for comfort and higher pressures to more quickly deal with events, etc.

In some implementations, the data associated with the user includes the age of the user; the age group of the user (e.g., less than 45, between 45 and 60, greater than 60, etc.); the gender of the user; the starting pressure for the user's use of the respiratory therapy system (e.g., the pressure that the respiratory therapy system will initially begin with at the beginning of the sleep session when the user initially dons the user interface and is likely still awake); the minimum therapy pressure for the user's use of the respiratory therapy system (e.g., the minimum working pressure of the respiratory therapy system when the user is asleep, which is generally prescribed by the user's healthcare provider); the user's baseline AHI; the user's baseline AHI group (e.g., minimum, mild, moderate, severe, unknown, etc.); the type of sleep test that the user has undergone (e.g., a home sleep test, a lab-based sleep test, etc.); the reason that the user will be using the respiratory therapy system (e.g., daytime sleepiness, restless sleep, concern from the user's bed partner, other health risk, other reasons, or any combinations of these reasons, etc.); the level of the user's sleepiness prior in the absence of using the respiratory therapy system (e.g., unknown, moderate, slightly, very, extremely, etc.); the type of user interface the user will use (e.g., full-face, nasal, nasal pillows, etc.); when the user initially started therapy with the respiratory therapy system (e.g., between 0 and 3 months ago, between 3 and 12 months ago, more than 1 year ago, more than 5 years ago, never, etc.); whether the user is or will be monitored by a healthcare provider during the user's use of the respiratory therapy system; a unique identifier of the respiratory therapy device and/or the user interface (such as a unique identification number); other data; or any combination of the above.

In some implementations, the data associated with the user includes data associated with sleep stages that the user spends time in during sleep sessions. For example, the data associated with the user could indicate on average how much time the user spends in various different sleep stages when asleep, what percentage of a sleep session is spent in each sleep stage on average, other types of data, or any combinations thereof.

The data may be received at any suitable location, such as the device or combination of devices that implement method 700. In some implementations, the data is received and stored in the memory device (such as memory device 114) of the system. In other implementations, the data is received and stored in other locations.

In step 720, an initial value of each of one or more parameters (also referred to herein as initial parameter values) of the respiratory therapy system is determined for use of the respiratory therapy system during a first period of time. The one or more parameters of the respiratory therapy system can include any parameter related to the user's use of the respiratory therapy system. The initial values of the parameters can be based at least in part on the received data (i.e., on the user data).

For example, the one or more parameters can include various settings of the respiratory therapy system. In some implementations, the parameters are associated with and/or impact a comfort level of the user during the sleep session. In these implementations, the parameters can include a pressure ramp setting, an event response setting, an expiratory pressure relief (EPR) setting, the temperature of the pressurized air delivered by the respiratory therapy system, the humidity of the pressurized air delivered by the respiratory therapy system, the standard pressure of the pressurized air during the sleep session, the temperature of the pressurized air, the temperature of the conduit connecting the user interface and the respiratory therapy device, the therapy mode of the respiratory therapy system (e.g., APAP, CPAP, BPAP, AUTO, etc.), the minimum pressure, the maximum pressure, the trigger time, and others.

Generally, the pressure ramp setting governs the gradual increase of the pressure of the pressurized air at the beginning of the sleep session (e.g., once the user initially dons the user interface during the sleep session). The pressure can increase from a starting pressure (e.g., 0) to some predetermined higher pressure. This allows the pressure to be gradually increased until the user is asleep, and prevents the user from experiencing higher pressures as they are trying to fall asleep. The pressure ramp setting generally includes multiple settings governing this increase. For example, a pressure ramp duration setting (which can also be referred to as a pressure ramp rate setting) defines how long it takes for the pressure to increase to the higher pressure (e.g., the rate at which the pressure is increased) once the ramp is implemented at the beginning of the sleep session. A final pressure setting defines the higher pressure that the pressure is increased to.

In some cases, the higher pressure that the pressure is increased to is the starting pressure that is used when the user is asleep during the sleep session. In other cases, the higher pressure is less than the starting pressure. In these cases, the pressure is maintained at the higher pressure until it is determined that the user is asleep, and then the pressure is increased to the starting pressure. In still other cases, the pressure is increased from the initial pressure to a pressure that is less than the higher pressure when the sleep session begins, and is then increased from that pressure to the higher pressure once it is determined that the user is asleep. In further cases, the pressure is increased to the higher pressure only after it is determined that the user is asleep. A higher pressure ramp duration value (e.g., a slower increase) and a lower final pressure value (e.g., an increase to a lower pressure) are generally more comfortable for the user while the user is still awake at the beginning of the sleep session, although they may be less optimal in treating the user's condition (e.g., OSA or SDB), as they may result in the respiratory therapy system taking a longer time to reach a pressure prescribed to treat the user's condition (e.g., a pressure prescribed to achieve a goal AHI during the sleep session).

The possible values of the pressure ramp setting may vary in different implementations. For example, in some implementations, the possible values of the pressure ramp setting arc “On” and “Off”. When set to On, a pressure ramp is implemented at the beginning of the sleep session with some predetermined duration and final pressure. When set to Off, no pressure ramp is implemented. In other implementations, the pressure ramp setting may additionally have a value of “Auto”. When set to Auto, a pressure ramp is implemented once it is determined that the user is asleep. Prior to the user being asleep, the Auto value can cause the pressure ramp setting to either not implement any pressure ramp, or implement a small pressure ramp. In other implementations, the possible values of the pressure ramp setting can be specific numerical values for the pressure ramp duration setting and the final pressure setting. In additional implementations, the possible values of the pressure ramp setting are specific numeral values for the pressure ramp setting, which each refer to a distinct combination of a pressure ramp duration value and a final pressure value.

The event response setting refers to how the respiratory therapy system adjusts the pressure of the pressurized air in response to the user experiencing an event. For example, if the user experiences an event (such as an apnea) during the sleep session, the pressure of the pressurize air may be gradually increased from its current pressure to some predetermined higher pressure, to aid in ending the event. Generally, the event response setting will define how the respiratory therapy system implements a pressure ramp (e.g., increase the pressure over some time period) in response to the event, where the pressure ramp is similar to the pressure ramp that may be implemented at the beginning of the sleep session in reference to the pressure ramp setting. However, in some implementations, the event response can additionally or alternatively define how other pressure changes besides a pressure ramp are implemented by the event response setting.

The event response setting can itself include multiple settings, including an event response duration setting and an event response final pressure setting. The event response duration setting defines the rate at which the pressure is changed (e.g., increased) in response to an event occurring. The event response final pressure setting defines the pressure (e.g., the higher pressure) that the pressure is changed (e.g., increased) to in response to an event. A higher event response duration value (e.g., a slower increase in a pressure ramp implemented to aid in mitigating the event) and a lower event response final pressure value (e.g., an increase to a lower pressure) are generally more comfortable for the user, although these values may be less effective at treating events.

Similar to the pressure ramp setting, the possible values of the event response setting may vary in different implementations. For example, in some implementations, the possible values of the event response setting are “Soft” and “Standard”. When set to Soft, the respiratory therapy system implements, in response to an event, a pressure ramp that is generally less aggressive (e.g., has a longer duration and/or a lower final pressure) than the pressure ramp implemented in response to an event when the value of the event response setting is set to Standard. In other implementations, the possible values of the event response setting can be specific numerical values for the event response duration setting and the event response final pressure setting. In additional implementations, the possible values of the event response setting are specific numeral values for the event response setting, which each refer to a distinct combination of an event response duration value and an event response final pressure value. In yet other implementations, the possible values of the event response setting are “On” and “Off”. When set to On, a predefined pressure ramp is implemented in response to an event occurring. When set to Off, no pressure ramp is implemented in response to an event occurring.

The pressure ramp setting and the event response setting are generally two different settings, even though they both can define a pressure ramp to be implemented by the respiratory therapy system. The pressure ramp setting generally defines a pressure ramp that can be implemented at the beginning (or toward the beginning) of a sleep session, that is designed to aid the user in falling asleep. The event response setting generally defines how the respiratory therapy system modifies the pressure of the pressurize air in response to the event, and in many cases defines a pressure ramp that is implemented by the respiratory therapy system in response to the event. The event response setting can define other pressure modification responses however.

As noted herein, the EPR setting allows for the respiratory therapy system to provide different pressures for the pressurized air depending on whether the user is current inhaling or exhaling. Thus, depending on the value of the EPR setting, there can be a pressure drop in the pressurized air between inspiration and expiration. When the EPR setting is active, the pressure of the pressurized air will be lower upon expiration as compared to inspiration. The possible values of the EPR may vary in different implementations. For example, in some implementations, the possible values of the EPR setting are “On” and “Off”. When set to Off, the pressure is not lowered during expiration, and when set to On, the pressure is lowered during expiration. In other implementations, the “On” setting is implemented as “On−1,” “On−2,” and “On−3.” On−2 implements a larger pressure drop during expiration than On−1, and On−3 implements a large pressure drop during expiration than On−2. In further implementations, the possible values for the EPR setting are specific numerical values that correspond to the amount that the pressure drops during expiration.

The parameters can also include parameters such as the light level in the room where the user uses the respiratory therapy system, the sound level in the room where the user uses the respiratory therapy system, and position the user is in when they use the respiratory therapy system, and others. Generally, all of the parameters can be adjusted in order to modify the user's experience with the respiratory therapy system. Some parameters may be adjusted by only the user, some parameters may be adjusted by only the user's care provider (e.g., doctor, caretaker, etc.), and some parameters may be adjusted by both the user and the user's care provider (and in some cases other people as well). For example, in some implementations, the pressure ramp setting, the event response setting, the EPR setting, the temperature of the pressurize air, and the humidity of the pressurize air can all be adjusted by both the user and the user's care provider. In some implementations, the set or starting pressure (e.g., the standard pressure of the pressurized air during the sleep session), the tidal volume, and other setting are adjustable only by the user's care provider.

In some implementations, one or more of the parameters may be associated with the user's comfort levels during their use of the respiratory therapy system, and can be adjusted to aid in adjusting the user's comfort level during the user's use of the respiratory therapy system. For example, a longer pressure duration and a lower final pressure during a pressure ramp arc generally more comfortable for the user. Thus, the value of the pressure ramp setting can be adjusted to aid in adjusting the user's comfort level. In another example, a lower pressure upon expiration can generally be more comfortable for the user. Thus, the value of the EPR setting can be adjusted to aid in adjusting the user's comfort level. Increased user comfort can in turn lead to greater compliance with the respiratory therapy system. The values of other parameters or settings can also be modified to aid in adjusting the user's comfort level and increase compliance with the respiratory therapy system.

In some implementations, the initial values of the parameters are generated by a first trained model, which may include one or more trained machine learning algorithms. The first model can receive as input any one or more of the types of data associated with the user discussed herein, and/or other data. The first model analyzes the data and outputs the initial values of the parameters. A variety of different models/algorithms can be used for the first model such as a casual interface recommendation algorithm, a content-based filtering recommendation algorithm, a reinforcement learning-based recommendation algorithm, a collaborative filtering recommendation algorithm, and others. In some implementations, the first model may actually be a combination of multiple different models and/or algorithms that are used in conjunction to determine the initial values of the parameters. For example, the first model may comprise separate models that each generate the initial value of distinct one or more of the parameters. In another example, the first model may comprise a first sub-model that receives the user data and generates intermediate data, and a second sub-model that receives the intermediate data (and/or the user data) and generates the initial values of the parameters.

The first model can be trained using training data generated from use of respiratory therapy systems by other users. The data that is input into the trained first model will change how various different settings of the parameters will impact the user's comfort levels (which as discussed, can be measured/estimated by looking at the user's compliance and/or other metrics). By training the first model based on how other users responded to different settings of the parameters, the trained first model can then analyze the data that is input for the user at issue to determine the initial values for the parameters. In some implementations, the trained first model is a causal inference model that determines the initial values of the parameters based on the user data.

In some implementations, the first model directly outputs the initial values of the parameters. In some of these implementations, the first model matches the user to one of a plurality of pre-existing user profiles based on the data that is input into the first model (e.g., the data associated with the user), and then determines the initial values of the parameters based at least in part on the matched user profile. The user profiles can be generated based on data received from a plurality of users that use a respiratory therapy system. The user profiles can be based on a variety of different factors, including user age, user gender, preferred user interface type, preferred comfort settings, clinical data, and other factors. Generally, data from a large number of users is used to establish the different user profiles, but data from any number of user profiles can be used. In some implementations, data from the user being matched to one of the user profiles was previously used to generate the user profiles. The user will generally be matched to the user profile with data most similar to the user. In some of these implementations (where the first model directly outputs the initial parameter values), each profile has a predefined set of initial values for each parameters (and/or ranges of initial values for the parameters) that the first model can select after determining which profile the use fits in. In others of these implementations (where the first model directly outputs the initial parameter values), the first model may determine the initial values of the parameters based on the profile, but without the initial values being predefined for the profile.

In other implementations, the first model does not directly output the initial values of the parameters, but instead only matches the user to one of the plurality of pre-existing user profiles. Once the first model matches the user to a profile, the predefined initial parameter values (e.g., values or ranges of values) for that profile can be manually selected (e.g., the user and/or a third party can update the settings of the respiratory therapy system with the initial values parameter values for the matched profile).

In still other implementations, the plurality of user profiles associated with the first model correspond to and/or simply are the various different combinations of all possible initial parameter values. For example, if two parameters are being adjusted, then a first profile could be parameter₁=initial value₁+parameter₂=initial value₁, a second profile could be parameter₁=initial value₁+parameter₂=initial value₂, a n^{th} profile could be parameter₁=initial valueₐ+parameter₂=initial value_{b}, etc. Thus, in any of the implementations described herein, when the first model matches the user to a user profile based at least in part on the user data, the first model may be matching the user to one of the distinct combination of initial parameter values based at least in part on the user data. Similarly, in any of the implementations described herein, when the first model determines a combination of initial parameter values from at least the user data, the first model may be matching the user to one of the user profiles based on at least the user data, where each user profile is that combination of initial parameter values. In general, in these implementations, there is not a separate determination of (i) which user profile the user matches and (ii) what the initial parameter values are. Instead, there may be a plurality of distinct combinations of initial parameter values, where each combination can be said to constitute a user profile. The first model is trained to analyze the user data and determine which combination of initial parameter values is best for the user, which in effect matches the user to one of the user profiles.

In step 730, usage data is received. The usage data is associated with the user's use of the respiratory therapy system during one or more sleep sessions in a first period of time, when the initial values of the parameters are used. The first period of time will generally include an initial period of n sleep session (e.g., n days). The usage data can be continually collected during the first period of time, intermittently collected during the first period of time, or collected only after the first period of time is complete. The usage data is generally received by and stored at the same location as the data received in step 710, which can be, for example, the memory device of the system implementing method 700, another location, etc.

The usage data includes information related to the user's use of the respiratory therapy system (also referred to herein as usage data) during this period of time when using the initial values of the parameters. In some implementations, the usage data includes the average user interface leak for the sleep sessions in the first period of time (e.g., the average volume of air per leak, the average volume of air leaked per sleep session, etc.); the standard deviation of the user interface leak for the sleep sessions in the first period of time; the average therapy pressure of the pressurized air for the sleep session in the first period of time; the standard deviation of the therapy pressure of the pressurized air for the sleep session in the first period of time; the average duration of use of the respiratory therapy system during the sleep sessions in the first period of time when the respiratory therapy system was actually used (e.g., the average minutes of use per sleep session for sleep sessions where the respiratory therapy system was used); the standard deviation of the duration of use of the respiratory therapy system during the sleep sessions in the first period of time when the respiratory therapy system was actually used; the average duration of use of the respiratory therapy system during all sleep sessions in the first period of time (e.g., the average minutes of use per sleep session for all sleep sessions); the standard deviation of the duration of use of the respiratory therapy system during all sleep sessions in the first period of time; the number of sleep sessions in the first period of time where the respiratory therapy system was used; the average number of times that the user interface was removed during sleep sessions in the first period of time; the standard deviation of the number of times that the user interface was removed during sleep sessions in the first period of time; the average residual AHI during the sleep sessions in the first period of time; the standard deviation of the residual AHI during the sleep sessions in the first period of time; the average residual apnea index (AI, which is generally the same as the AHI but does not include hypopnea events) during the sleep sessions in the first period of time; the standard deviation of the residual AI during the sleep sessions in the first period of time; the average number of RERA events during sleep sessions in the first period of time; the standard deviation of the number of RERA events during sleep session in the first period of time; other data; or any combination of the above. Any of the above averages and standard deviations can be determined across any suitable amount of time, including per second, per minute, per hour, per sleep session, and/or other amounts of time.

The usage data can also include data associated with sleep stages that the user spent time in during the sleep sessions in the first period of time when the initial values of the parameters were used. The data can include an amount of time the user spent in each of a plurality of sleep stages during each sleep session, the average amount of time spent in each of the plurality of sleep stages during the sleep sessions of the first period of time (which may be all of the sleep sessions and/or all of the sleep sessions where the respiratory therapy system was used), the standard deviation of the amount of time spent in each of the plurality of sleep stages during the sleep sessions of the first period of time, the sleep stage the most time was spent in for each sleep session (which may be all of the sleep sessions and/or all of the sleep sessions where the respiratory therapy system was used), other types of data, or any combination thereof.

The usage data can also include subjective input from the user, which can generally include any information that the user provides related to their use of the respiratory therapy system during the first period of time with the initial values of the parameters. For example, the subjective input can include user-submitted information associated with the comfort level of the user interface worn during the sleep sessions in the first period of time, the comfort level of the user's breathing during the sleep sessions in the first period of time (e.g., whether the user had any difficulty breathing in and out), an amount of restlessness the user experienced during the first period of time, or any combination thereof. The user can provide the subjective input in any suitable manner. For example, the user can provide the subjective input using a mobile device such as a smart phone or a tablet computer. The user can also use an external computing device such as a laptop computer or a desktop computer. The user can further use the respiratory therapy system itself, if the respiratory therapy system can accept user input. Generally, the user can use any user device (such as user device 170) of the system.

The usage data can be received in a variety of different manners. In some implementations, the usage data for the one or more sleep sessions in the first period of time is received only after all of the sleep session in the first period of time are completed. In other implementations, the usage data for the first period of time is received continually during the first period of time. In these implementations, the usage data for the first period of time generally includes multiple portions of usage data, where each portion of usage data corresponds to a respective one of the one or more sleep sessions in the first period of time. Each portion of usage data may be received after the completion of its respective sleep session, or may be received continually during its respective sleep session. In either case, the usage data for the first period of time is received continually during the first period of time.

At step 740, recommended values for the one or more parameters of the respiratory therapy system (also referred to here as recommended parameter values) are generated. The recommended values for the parameters can be based at least in part on the usage data, optionally in conjunction with the user data and/or other types of data. As discussed herein, the recommended values for the parameters will generally be the parameter values that are determined to have the best probability of increasing the user's compliance (or have the best probability of optimizing some other quantity or parameter, as discussed herein) for the user, and can be used during the user's use of the respiratory therapy system during a second period of time.

In some implementations, the first period of time includes a predetermined number of sleep sessions (e.g., a predetermined number of days/nights). In these implementations, the recommended values for the one or more parameters can be generated after completion of the predetermined number of sleep sessions. However, initial recommended values for the parameters could be generated after completion of the first sleep session, and then updated after completion of each subsequent sleep session in the first period of time, until the predetermined number of sleep sessions have been completed.

In other implementations, the first period of time can include a variable number of sleep sessions. In these implementations, the recommended values for the parameters can be generated after any number of sleep sessions within the first period of time, based on a variety of different factors. The recommended values can be generated only after the final sleep session of the first period of time (whichever sleep session that happens to be), or initial recommended values can be generated after the first sleep session and then updated after completion of each subsequent sleep session, until the final sleep sessions (whichever sleep session that happens to be) is completed. In some cases, the recommended values for the parameters are generated if it is determined that the user's compliance with the respiratory therapy system during the first period of time fails to satisfy a predetermined threshold after a given sleep session. In other cases, the final recommended values are generated if it is determined that the difference between the current value of at least one of the parameters and its continually update recommended value satisfies a predetermined threshold.

In further cases, the recommended values can be generated based on the subjective input of the user. For example, if the user indicates that the current values of the parameters are undesirable for some reason (e.g., the user indicating that they are uncomfortable during the sleep session), the recommended values can be generated. The user could be asked to provide their subjective input at any time. For example, the user could be asked to provide their subjective input about their use of the respiratory therapy system every n sleep session (which in most cases will be equal to every n days). The user could also proactively provide the subjective input.

In some cases, the recommended values can be generated if the user switches to a different user interface. The recommended values for the parameters may be better suited to the new user interface type, and will generally be designed to ensure that the user maintains (or improves) compliance with the new user interface type. For example, the usage data may indicate that the user has switched from a full face mask to nasal pillows. When this change occurs, recommended values for the parameters that work better with nasal pillows can be generated.

Similar to the initial values, in some implementations the recommended parameter values are generated by a second trained model, which may include one or more trained machine learning algorithms. The second model can receive as input any of the one or more types of usage data. The second model can also receive any of the one or more types of user data, the initial parameter values, other data, or any combination thereof. The second model analyzes at least the usage data and outputs the recommended values of the parameters. A variety of different models/algorithms can be used for the second model such as a casual interface recommendation algorithm, a content-based filtering recommendation algorithm, a reinforcement learning-based recommendation algorithm, a collaborative filtering recommendation algorithm, and others. In some implementations, the second model may actually be a combination of multiple different models and/or algorithms that are used in conjunction to determine the recommended values of the parameters. For example, the second model may comprise separate models that each generate the recommended value of a distinct one or more of the parameters. In another example, the second model may comprise a first sub-model that receives the usage data (and/or any other data) and generates intermediate data, and a second sub-model that receives the intermediate data (and/or the usage data) and generates the recommended values of the parameters. Moreover, in some implementations, the second model may be the same model as the first model, such that a single model generates both the initial values and the recommended values. In other implementations, the first model and the second model are distinct models (even if they are the same type of model).

The second model can be trained using training data generated from use of respiratory therapy systems by other users, similar to the first model. The data that is input into the trained second model will change how various different settings of the parameters will impact the user's comfort levels (which as discussed, can be measured/estimated by looking at the user's compliance and/or other metrics). By training the second model based on how other users responded to different settings of the parameters, the trained second model can then analyze the data that is input for the user at issue to determine the recommended values for the parameters. In some implementations, the trained second model is a causal inference model that determines the recommended values of the parameters based at least in part on the usage data.

The second model determines the recommended values of the parameters of the parameters based on the usage data, or based on the usage data and one or more other types of data, which can include the user data (which was input into the first model), the initial parameter values, the user profile to which the user was matched to by the first model, or any combination thereof. For example, in some implementations, the second model matches the user to a user profile (which may or may not be the same as the user profile determined by the first model) based on the user data, and then determines the recommended parameter values based on the user profile and the usage data. In some cases, each user profile can have a predefined recommended value (or range of values) for each parameter, that is then adjusted by the usage data. In other cases, each user profile has, for each parameter, a plurality of potential recommended values (or ranges of values). After the user profile is determined based on the user data, the recommended value of each parameter is selected from the plurality of potential recommended values based on the usage data. In other implementations, the second model matches the user to a user profile based on the user data and the usage data. The user profile determined by the second model may have predefined recommended values for each parameter that can then be output by the second model. In additional implementations, the second model may also analyze the initial parameter values, and use the initial parameter values in conjunction with the usage data (and in some cases also in conjunction with the user data) to determine the user profile.

In some implementations, the second model does not directly match the user to any user profile. Instead, the second model receives the usage data, and an indication of which user profile the user was matched to by the first model (which is based at least in part on the user data). The second model can then determine the recommended parameter values based on the usage data and the matched user profile. Thus, the second model can analyze how a certain user profile reacted with the initial parameter values, and set the recommended parameter values accordingly.

In some implementations, the user data affects the determination of the final parameter values only insofar as that the final parameter values are based on the usage data and the user profile/set of initial parameter values the user was given based on the user data. Thus, two users whose user data resulted in them receiving the same initial parameter values would generally have the same final parameter values, if those two users had the same or similar usage data. However, in other implementations, the final parameter values are based on the usage data and the user data itself, instead of the user profile/set of initial parameter values dictated by the user data. Thus, two users whose user data resulted in them receiving the same initial parameter values could have different final parameter values—despite having the same or similar usage data—if their user data was different enough.

In some implementations, the second model does not directly output the recommended values of the parameters, but instead only matches the user to one of the plurality of user profiles. Once the second model matches the user to a profile, predefined recommended parameter values (e.g., values or ranges of values) for that profile can be manually selected (e.g., the user and/or a third party can update the settings of the respiratory therapy system with the recommended values parameter values for the matched profile).

Similar to the first model, in some implementations the plurality of user profiles associated with the second model are simply the various different combinations of all possible recommended parameter values. Thus, in any of the implementations described herein, when the second model matches the user to a user profile based at least in part on the usage data, the second model may be matching the user to one of the distinct combination of recommended parameter values based on the usage data, the usage data and the user data, or the usage data and any other combination of data. Similarly, in any of the implementations described herein, when the second model determines a combination of recommended parameter values from at least the usage data, the second model may be matching the user to one of the user profiles based on at least the usage data, where each user profile is that combination of recommended parameter values. In general, in these implementations, there is not a separate determination of (i) which user profile the user matches and (ii) what the recommended parameter values are. Instead, the second model is trained to analyze the usage data and/or the user data to determine which combination of recommended parameter values (each of which can be said to constitute a user profile) is best for the user, which in effect matches the user to one of the user profiles.

In some implementations, the inputs to the first model and or the second model include the specific parameters that are able to be adjusted for that user. For example, a certain user may not have the ability to adjust the light and/or sound levels in their room, or may indicate that they prefer a lower pressure ramp duration. Thus, the inputs into the first model and/or the second model can include these preferences, so that the first model and/or the second model do not output initial values or recommended values for a parameter that the user is unable or unwilling to comply with.

In some implementations, the recommended values for the parameters can be transmitted to the user, a care provider of the user (e.g., a doctor or caretaker), or both. The user and/or the care provider may need to manually update the values of the parameters to the recommended values, or the parameters can be updated automatically. In some cases, the recommended values are presented to the user on an application interface (e.g., a display screen). The application interface may be located on the respiratory therapy device, a mobile device, an external computing device, or any other suitable device. The user could also be presented with an option to accept or decline the recommended values. In some cases, multiple recommended values could be generated, and the selection of which recommend value can be presented to the user. In these cases, the selection of a recommended value may include an option to increase or decrease the value. The user could also be provided with an option to accept or decline the recommended values, and/or to suggest their own values for various settings.

In some implementations, generating the recommended values can be based on the degradation of the user interface. For example, a sensor (e.g., a camera, an acoustic sensor, etc.) can be used to determine whether the user interface is degraded beyond an acceptable level. The degree of degradation can be determined visually, for example using a camera that generates visual evidence of the degradation. The degree of degradation can also be determined using an acoustic sensor that is used to detect the sound of air leaking from the user interface. If the degree of degradation of the user interface satisfies a predetermined threshold (e.g., if the user interface has degraded a certain amount), a recommendation to obtain a new user interface (and/or update the type of user interface) can be generated and transmitted to the user. The system can optionally submit a resupply order if the user so chooses.

In some implementations, method 700 can also include optional steps 750, 760, and 770. At optional step 750, the one or more parameters are updated to their recommended values for use with the respiratory therapy system during the second period of time, and the user uses the respiratory therapy system for one or more sleep session in the second period of time with the recommended values. At optional step 760, subsequent usage data is received. The subsequent usage data is associated with the user's use of the respiratory therapy system during the one or more sleep sessions of the second period of time. At optional step 770, subsequent recommended values for the one or more parameters of the respiratory therapy system are generated. The subsequent recommended values can be based at least in part on the user data, the subsequent usage data, or both. The subsequent recommended values can be used for the parameters during use of the respiratory therapy system in a third period of time after the second period of time.

The subsequent usage data can include similar information as the usage data, except that it is related to use of the respiratory therapy system using the recommended values of the parameters. The subsequent usage data can be received continually during the second period of time (e.g., after each sleep session of the second period of time or continually during the sleep sessions of the second period of time), or after all of the sleep sessions in the second period of time have been completed. Similar to the first period of time, the second period of time can have a predetermined or variable number of sleep sessions, and the subsequent recommended values can be generated after all of the predetermined number of sleep sessions have been completed, or after a certain variable number of sleep sessions have been completed.

Generally, the values of the parameters can be continually and/or dynamically updated to improve the user's comfort and/or compliance with the respiratory therapy system. For example, new recommended values for the parameters can be generated (or continually updated) periodically to provide the user with the best possible experience. New recommended values can be updated every n sleep sessions (or every n days). New recommended values can also be updated whenever the usage data indicates that new recommended values are needed, for example of the user's compliance is decreasing or the subjective user input indicates that the user is not satisfied or comfortable. The user can provide their subjective input whenever they want, or the system could periodically ask the user to provide their subjective input.

In some of the implementations discussed herein, the first and second models directly output specific combinations of settings, and are generally what is referred to prescriptive models. Prescriptive models search for the best or optimal output given the inputs. In these implementations, the models do not compare a user to past users, or compare user data to user data from past users. Instead, the models are trained to determine the best treatment option given the user data (and in some cases also usage data) that is input into the model. These models are trained on a large amount of observational data that is used to form the training data sets. This observational data includes information about past patients, the treatments that the patients used (e.g., the combinations of settings used with the respiratory therapy systems), and outcome data (e.g., an indication of whether the patient complied with a predetermined standard of compliance). However, this observational data will generally come with a significant amount of bias. For example, the same patient may be prescribed to different treatments (e.g., combinations of settings) by two different doctors, even though the data about the patient is the same. In another example, one doctor may have a tendency to primarily prescribe one treatment to their patients regardless of differences between their patients, while another doctor may have a tendency to prescribe more varied treatments to their patients.

These biases in the observational data can cause the models to be overfit to the training data, which can in turn result in the models outputting less than optimal combinations of settings (and/or probabilities that indicate a less than optimal combination of settings should be used). To remove this bias, in some implementations, method 700 can utilize doubly robust learner techniques for the first model that is used to generate the initial parameter values at step 720, and/or for the second model used to generate the recommended parameter values at step 740. In these implementations, the first model and the second model are multi-stage models that combine a propensity model, an outcome model, and then a final model that utilizes the outputs of the propensity model and the outcome model.

The propensity model is trained to predict the probability of receiving a certain treatment (e.g., a specific combination of settings) given the user data. The propensity model reduces (and/or removes) the bias in the observational data so that the more closely approximates a randomized controlled trial (which is controlled so there is little or no bias in the data). The outcome model predicts outcomes for specific combinations of settings and user data. This predicted outcome will generally be in the form of a probability that the predetermined compliance threshold will be met given the specific combination of settings and the user data. The final model utilizes the outputs of the propensity model and the outcome model to predict the outcome for a specific set of user data. Similar to the outcome model, predicted outcome of the final model will generally be in the form of a probability that the predetermined compliance threshold will be met, but given only the user data, and not a specific combination of settings.

In some implementations, when the first model and the second model are used with a specific patient, the propensity model receives the user data associated with that user, and outputs a propensity score for each individual combination of settings. This propensity score is indicative of the likelihood that each of the individual combination of settings would have been prescribed to the user by their doctor, given the specific user data. The propensity score can be expressed as Pr [T=t|X], which is the probability of being prescribed a specific treatment T (e.g., a combination of settings) given the user data X.

The outcome model receives the user data for the user, and outputs conditional compliance probabilities for that specific user data conditioned on using each of the different combinations of settings. These conditional probabilities can be expressed as E [Y|X, T], which is the expected value of the outcome Y (e.g., a probability between 0 and 1 of satisfying the compliance threshold) given user data X and treatment T.

Finally, the propensity scores and the conditional probabilities are combined in a final model that receives the user data, and outputs the difference between (i) the compliance probability for one of the combination of settings that is considered the default combination of settings (which may also be referred to as the conditional average treatment effect), and (ii) the compliance probability of each of the possible different combinations of settings (besides the default) given that user data. The combination of settings with the highest difference in compliance probability relative to the default combination of settings can then be selected for the user. In some cases, the final model predicts the conditional average treatment effect by regressing a pseudo-outcome Y_{DR} on user data X directly, where:

$${Y}_{DR} = ( \frac{W}{\hat{π} ( X )} - \frac{1 - W}{1 - \hat{π} ( X )} ) Y + [ ( 1 - \frac{W}{\hat{π} ( X )} ) {\hat{μ}}_{1} ( X ) - ( 1 - \frac{1 - W}{1 - \hat{π} ( X )} ) {\hat{μ}}_{0} ( X ) ] .$$

In this equation, Y_{DR} is the difference in compliance probability between the default combination of settings and a specific combination of settings W; \hat{π}(X) is the propensity score (determined by the propensity model) for the combination of settings W based on the user data X; Y is the real outcome; \hat{μ}₀ is the predicted outcome of the default combination of settings given the user data X determined by the outcome model; and \hat{μ}₁ is the predicted outcome of the combination of settings W determined by the outcome model.

In some cases, the final model is used to directly calculate the difference in compliance probability for a given set of settings W and user data X. In these cases, the above formula for Y_{DR} is used with an assumption that the outcome for settings W will be satisfaction of the compliance threshold, and thus the value of the real outcome Y is set as 1. In other cases, the final model is used to predict the difference in compliance probability, and this is trained on the training data. In these cases, for a given training datapoint comprising a patient with user data X that used user settings W, the value of the real outcome Y is set according to whether the patient with user data X actually satisfied the compliance threshold with user settings W. If the compliance threshold was satisfied, Y is set as 1, and if the compliance threshold was not satisfied Y is set as 0.

Both the first model used to generate the initial parameter values in step 720 and the second model used to generate the recommended parameter values in step 740 can have this multi-model form. The primary (and sometimes only) difference between the first model and the second model is that in the first model, the X variable contains only the user data, and in the second model, the X variable contains both the user data and the usage data.

In one example, the data set used to train and test the first and second models included data associated with 492,076 patients. For each patient, the data included values for each individual feature in the user data (e.g., age, gender, user interface type, baseline AHI group, etc.), the specific combination of settings used by the user, an indication of whether the user satisfied the predetermined compliance threshold, and values for each individual feature in the usage data (e.g., average user interface leak, average duration of use per night, average residual AHI, etc.). In this example, the patients were split into a training group that included 60% of the patients, a validation group that included 15% of the patients, and a testing group that include 25% of the patients. However, other splits and total numbers of patients may be used to train models such as those described herein.

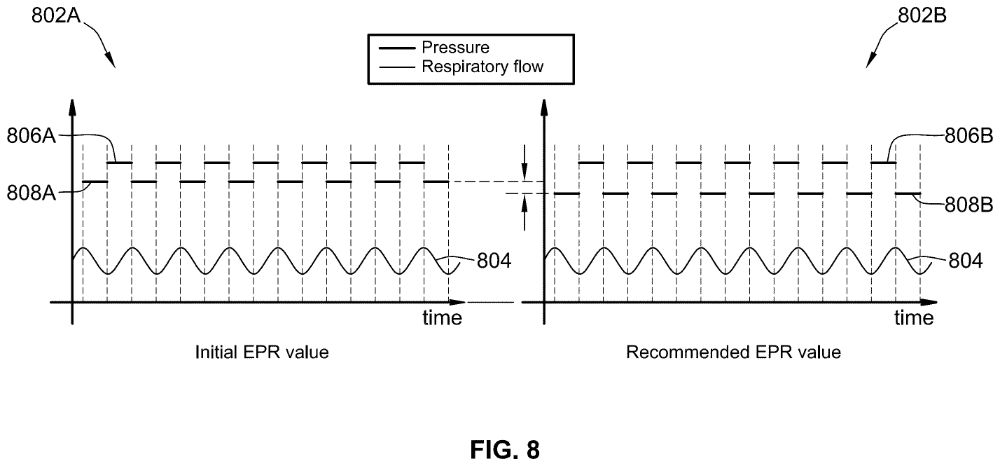

FIG. 8 includes two plots showing one implementation of method 700, according to aspects of the present disclosure. The left side plot 802A shows the use of a respiratory therapy system with an initial value of the EPR setting. Plot 802A shows a respiratory flow signal 804, an inspiration pressure signal 806A, and an expiration pressure signal 808A. The pressure of the pressurized air during inspiration (e.g., when the respiratory flow signal 804 is increasing or when the slope of the respiratory flow signal 804 is positive) is higher than the pressure of the pressurized air during expiration (e.g., when the respiratory flow signal 804 is decreasing or when the slope of the respiratory flow signal 804 is negative). This difference in pressures can be seen by comparing the inspiration pressure signal 806A and the expiration pressure signal 808A. The lower pressure during expiration as compared to inspiration is designed to provide a more comfortable experience to the user.

The right side plot 802B shows the result of the changing the value of the EPR setting to its recommended value, for example using the techniques of method 700. Plot 802B includes the same respiratory flow signal 804 as the left side plot 802A, an inspiration pressure signal 806B, and an expiration pressure signal 808B. However, the value of the expiration pressure signal 808B has been modified to be less than the value of the expiration pressure signal 808A, resulting in a lower pressure during expiration. This greater difference in pressures between the inspiration pressure signal 806B and the expiration pressure signal 808B may be more comfortable for the user—even if it is less effective at mitigating events during the sleep session—and may result in increased user compliance with the respiratory therapy system.

In FIG. 8, the inspiration pressure signals 806A and 806B generally have the same value. Thus, the pressure of the pressurized air during inspiration is generally the same in both cases. However, the recommended value of the EPR setting could include a change in the value of the inspiration pressure, in addition to or as an alternative to the change in the value of the expiration pressure. Thus, the inspiration pressure signal 806B in plot 802B could in some implementations be higher or lower than that shown in FIG. 8.

While FIG. 8 shows plots illustrating the result of the recommended value of the EPR setting being different than the initial value, any parameter related to the use of the respiratory therapy system can have its value changed using the techniques of method 700. For example, while the plots of FIGS. 6A and 6B are used herein to illustrate implementations of method 500, modifications of the values of a pressure ramp setting according to method 700 can result in similar changes as those shown in FIGS. 6A and 6B.

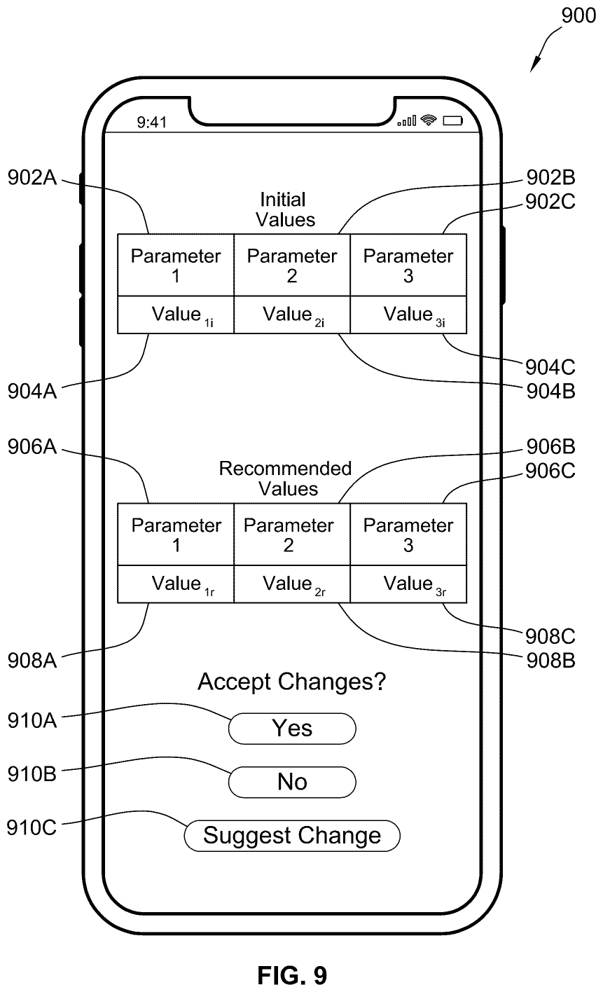

FIG. 9 shows an example of an interface of a user device 900 being used to transmit recommended values to the user and/or the user's care provider, and to provide the user with an option to accept or decline the recommended values. In FIG. 9, the user device 900 is a smart phone. However, any type of user device (tablet computer, laptop computer, smartwatch, etc.) can be used to present the user with the recommended values. The recommended values can also be presented on an application interface of the respiratory therapy device of the respiratory therapy system.

The user device 900 shows a table with initial values for the parameters in questions. In the illustrated implementation, the table includes cells 902A, 902B, and 902C that show the name of the parameters in questions, and cells 904A, 904B, and 904C that show the initial value of each of the parameters. For illustrative purposes, the generic names “Parameter 1,” “Parameter 2,” and “Parameter 3” are used to show the parameters being modified, and the generic initial values “Value₁ᵢ,” “Value₂ᵢ,” and “Value₃ᵢ” are used to show the initial values of the parameters.

The user device 900 shows an additional table with the recommended values for the parameters to be modified. This table includes cells 906A, 906B, and 906C that show the names of the parameters, and cells 908A, 908B, and 908C that show the recommended values of the parameters. Again, for illustrative purposes, the generic names are used to show the parameters being modified, and the generic recommended values “Value₁ᵣ,” “Value₂ᵣ,” and “Value₃ᵣ” are used to show the recommended values of the parameters. In some implementations, the recommended values are generated using method 700. Thus, the recommended values can be generated based on usage data of the respiratory therapy system (which may include subjective user input) and the user profile to which the user has been matched.

The user device 900 finally shows the words “Accept Changes? above three user-selectable icons 910A, 910B, and 910C. User-selectable icon 910A includes the text “Yes,” and can be selected by the user to accept the recommended values and use the recommended values during one or more subsequent sleep sessions. User-selectable icon 910B includes the text “No,” and can be selected by the user to decline the recommended values and continue to use the initial values during one or more subsequent sleep sessions. User-selectable icon 910C includes the text “Suggest Changes,” and can be selected by the user if the user wishes to modify the recommended values in some manner. In some implementations, in response to the user selecting icon 910C, the application interface allows the users to directly input the user's preferred values via an interactive text box. In other implementations, the application interface allows the user to increase or decrease the recommended values, for example by displaying user-selectable icons corresponding to an increase and a decrease. In further implementations, instead of showing user-selectable icon 910C, the application interface shows an interactive text box and/or the increase and decrease icons.

Thus, the recommended values for the parameters can be transmitted directly to the user and/or the user's care provider after they are generated. In some implementations, the parameters being modified include a specific combination of two or more parameters that are associated with and/or impact (directly or indirectly) the comfort level of the user. In one example, the parameters being modified include any two or more parameters selected from at least the following list: (i) a pressure ramp setting of the respiratory therapy system, (ii) an event response setting of the respiratory therapy system, (iii) an expiratory pressure relief setting of the respiratory therapy system, (iv) a temperature of pressurized air delivered by the respiratory therapy system, and (v) a humidity of the pressurized air delivered by the respiratory therapy system.

In some implementations, the combination of two or more parameters is selected based on the likelihood of success of the recommended values, weighted based on the user profile that the user is matched into. In other implementations, the combination of two or more parameters is selected based on balancing the effect of each of the parameters on the comfort of the user that belongs to a particular user profile. In general, the combination of two or more parameters may be selected or customized based on any requirements, such as improving the user's compliance with a prescribed use of the respiratory therapy system, increasing the user's comfort level, or other requirements.

As discussed herein, in some implementations the recommended values of the one or more parameters are the values of the parameters that are estimated to improve and/or maximize compliance, however compliance is designed. However, the recommended values of the parameters can also be values that are estimated to affect other variables as well. In some implementations, the recommended parameter values are the values estimated to improve and/or maximize a self-reported comfort score from the user. In some implementations, the recommended parameter values are the values estimated to increase and/or maximize the amount of time spent at or below a certain therapy pressure while still achieving a target AHI. In some implementations, the recommended parameter values are the values estimated to increase and/or maximize the probability that the user will continue to use the respiratory therapy system after a trial period of using the respiratory therapy system. In some implementations, the recommended parameter values are the values estimated to increase and/or maximize the number of days (or sleep sessions) that the user will use the respiratory therapy system within a certain time period. In some implementations, the recommended parameter values are the values estimated to increase and/or maximize the amount of time the user will use the respiratory therapy system during one or more sleep sessions. In some implementations, the recommended parameter values are the values estimated to decrease and/or minimize the number of disruptions to the use of the respiratory therapy system (such as the user removing the user interface, the user interface inadvertently being removed, the respiratory therapy device becoming disconnected, the conduit becoming disconnected, the user interface becoming disconnected, some other component becoming disconnected, etc.). In some implementations, the recommended parameter values are the values estimated to decrease and/or minimize the number of user interface on-off events during the sleep session.

In general, the recommended parameter values are the values having the highest probability (or estimated to have the highest probability) of resulting in the user satisfying a threshold for a use metric associated with the user's use of the respiratory therapy system. The use metric is generally any metric that can be used to measure the user's use of the respiratory therapy system. The use metric could be the compliance with a prescribed plan of use of the respiratory therapy system, in which case satisfying the threshold for the use metric includes satisfying the minimum requirements of the prescribed plan of use of the respiratory therapy system (e.g., using the respiratory therapy system at least x hours per sleep session, using the respiratory therapy system for at least y sleep sessions within the first z months of use, etc.). In other cases, satisfying the threshold for the use metric includes achieving at least a minimum acceptable value for some metric (e.g., achieving some minimum average amount of time spent using the respiratory therapy system per sleep session). In further cases, satisfying the threshold for the use metric includes not exceeding a maximum acceptable value for some metric (e.g., not exceeding a maximum average number of events per sleep session).

In some implementations, the first model is trained to determine, for each distinct combination of initial parameter values (where each distinct combination can be said to constitute and/or correspond to a user profile), the probability that that combination will result in the user satisfying the threshold for a use metric during the first period of time. This determination can be based at least in part on the user data, e.g., the first model can be trained to determine how each combination of initial parameter values will impact the usage of a user that corresponds to the user data. The first model can then select the combination of initial parameter values (e.g., one of the user profiles) that has the highest probability among all of the possible combinations of initial parameter values.

In some of these implementations, the second model is trained to generate the recommended parameter values based on the user data and/or the usage data (which may include information on how the user used the respiratory therapy system with the selected combination of initial parameter values (e.g., the selected user profile)). For example, based on the user data and/or the usage data, the second model may determine, for respective combination of recommended parameter values, the probability that the respective combination of recommended parameter values will result in the user satisfying the threshold for the use metric during the second time period. The combination of recommended parameter values with the highest probability can then be selected (e.g., the parameter values can be modified from the initial parameter values to the recommended parameter values). In some cases, the selected combination of recommended parameter values maximizes the probability that the user will satisfy the threshold for the use metric during the second time period. In some cases, the selected combination of recommended parameter values will increase the probability that the threshold for the use metric will be satisfied during the second period of time as compared to the first period of time.

In some implementations, method 700 can also include selecting the parameters to be optimized. For example, during the first period of time, the respiratory therapy system can be used while a plurality of parameters each have an initial value. After the first period of time one or more of these parameters can be selected to have their values modified, and the recommended values for those selected parameters can be generated. The selection of the parameters to modify and the generation of the recommendation can be based at least in part on usage data and/or the user profile to which the user has been matched.

Generally, methods 500 and 700 can be implemented using a system having a control system with one or more processors, and a memory device storing machine readable instructions. The control system can be coupled to the memory device, and methods 500 and 700 can be implemented when the machine readable instructions are executed by at least one of the processors of the control system. Methods 500 and 700 can also be implemented using a computer program product (such as a non-transitory computer readable medium) comprising instructions that when executed by a computer, cause the computer to carry out the steps of methods 500 and 700.

One or more elements or aspects or steps, or any portion(s) thereof, from one or more of any of claims 1 to 103 below can be combined with one or more elements or aspects or steps, or any portion(s) thereof, from one or more of any of the other claims 1 to 103 or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations or alternative implementations according to aspects of the present disclosure may combine any number of features from any of the implementations described herein, such as, for example, in the alternative implementations described below.

1. A method of optimizing a plurality of parameters of a respiratory therapy system, the method comprising:
receiving user data associated with a user of the respiratory therapy system;
based at least in part on the user data, determining an initial value of each of the plurality of parameters, each of the plurality of parameters being associated with a comfort level of the user;
receiving usage data associated with use of the respiratory therapy system during one or more sleep sessions in a first period of time in which each of the plurality of parameters has its initial value; and
based at least in part on the user data and the usage data, generating a recommended value of each of the plurality of parameters for use of the respiratory therapy system during a second period of time after the first period of time.

2-5. (canceled)

6. The method of claim 1, wherein generating the recommended values of each of the plurality of parameters is based on a probability that the recommended values of each of the plurality of parameters will result in the user satisfying a threshold for a use metric during the second period of time, the use metric being associated with use of the respiratory therapy system by the user.

7. The method of claim 6, wherein the use metric is compliance with a plan of use of the respiratory therapy system by the user, and wherein the user satisfying the threshold for the use metric includes the user satisfying one or more minimum requirements of the plan of use during the second period of time.

8. The method of claim 6, wherein the user satisfying the threshold for the use metric includes the user (i) achieving at least a minimum acceptable value of the use metric during the one or more sleep sessions in the second period of time or (ii) not exceeding a maximum acceptable value of the use metric during the one or more sleep sessions in the second period of time.

9-14. (canceled)

15. The method of claim 1, wherein generating the initial value of each of the plurality of parameters includes:
determining, for each respective combination of a plurality of combinations of initial values of the plurality of parameters, a probability that the respective combination will cause the user to satisfy a threshold for a use metric associated with the use of the respiratory therapy system by the user, the determining being based at least in part on the user data; and
selecting a combination of initial values of the plurality of parameters having a highest probability among the plurality of combinations of resulting in the user satisfying the threshold for the use metric.

16. The method of claim 15, wherein the recommended value of each of the plurality of parameters is generated based at least in part on the user data, the usage data, the selected combination of initial values of the plurality of parameters, or any combination thereof.

17. The method of claim 1, wherein generating the recommended value of each of the plurality of parameters includes:
determining, for each respective combination of a plurality of combinations of initial values of the plurality of parameters, a probability that the respective combination of initial values of the plurality of parameters will cause the user to satisfy a threshold for a use metric during the first period of time, the use metric being associated with the use of the respiratory therapy system by the user, the determining being based at least in part on the user data;
selecting a combination of initial values of the plurality of parameters having a highest probability among the plurality of combinations of initial values of the plurality of parameters of resulting in the user satisfying the threshold for the use metric during the first period of time;
determining, for each respective combination of a plurality of combinations of recommended values of the plurality of parameter, a probability that the respective combination of initial values of the plurality of parameters will result in the user satisfying a threshold for a use metric during the first period of time, the determining being based at least in part on the user data and the usage data;
selecting a combination of recommended values of the plurality of parameters having a highest probability among the plurality of combinations of recommended values of the plurality of parameters of resulting in the user satisfying the threshold for the use metric during the second period of time.

18. The method of claim 17, wherein the selected combination of recommended values of the plurality of parameters is configured to (i) maximize the probability of the user satisfying the threshold for the use metric during the second period of time or (ii) increase the probability of the user satisfying the threshold for the use metric during the second period of time as compared to the first period of time.

19. (canceled)

20. The method of claim 1, further comprising transmitting the generated recommended value to (i) the user, (ii) a care provider of the user, or (iii) both.

21. The method of claim 1, further comprising presenting the generated recommended value on an application interface located on (i) a respiratory therapy device of the respiratory therapy system, (ii) a mobile device, (iii) an external computing device, or (iv) any combination thereof.

22. The method of claim 21, wherein the application interface provides an option to (i) accept or decline, and (ii) increase or decrease, if the generated recommended value is accepted.

23. The method of claim 1, wherein the user data includes (i) an age of the user, (ii) a gender of the user, (iii) clinical data of the user, (iv) a user interface type of the respiratory therapy system used by the user, (v) a test type used by the user, or (vi) any combination thereof.

24. The method of claim 23, wherein the clinical data of the user includes (i) an Apnea-Hypopnea Index of the user, (ii) a prescribed respiratory pressure for the user, (iii) a sleepiness/restlessness score of the user, (iv) reasons for therapy of the user, or (v) any combination thereof.

25-26. (canceled)

27. The method of claim 1, wherein the plurality of parameters includes two or more of (i) a pressure ramp setting of the respiratory therapy system, (ii) an event response setting of the respiratory therapy system, (iii) an expiratory pressure relief setting of the respiratory therapy system, (iv) a temperature of pressurized air delivered by the respiratory therapy system, and (v) a humidity of the pressurized air.

28. The method of claim 1, wherein the usage data includes information associated with (i) a frequency of use of the respiratory therapy system, (ii) pressurized air delivered by the respiratory therapy system, (iii) one or more leaks associated with the pressurized air, (iv) subjective input received from the user, or (v) any combination thereof.

29. The method of claim 28, wherein the subjective input received from the user includes information associated with (i) a comfort level of a user interface worn by the user during use of the respiratory therapy system, (ii) a comfort level of the user's breathing during use of the respiratory therapy system, (iii) an amount of restlessness the user experienced during use of the respiratory therapy system, or (iv) any combination thereof.

30. The method of claim 1, wherein the generating the recommended value of the plurality of parameters is performed using (i) a causal inference recommendation algorithm, (ii) a content-based filtering recommendation algorithm, (iii) a reinforcement learning-based recommendation algorithm, or (iv) a combination of both.

31-32. (canceled)

33. The method of claim 1, wherein a first parameter of the plurality of parameters is an event response setting of the respiratory therapy system, and wherein a second parameter of the plurality of parameters is an expiratory pressure relief setting of the respiratory therapy system.

34. The method of claim 33, wherein the event response setting defines a duration and a final pressure of a pressure ramp implemented by the respiratory therapy system in response to the user experiencing an event during the one or more sleep sessions, and wherein the expiratory pressure relief setting defines a decrease in a pressure of pressurized air delivered by the respiratory therapy system to the user during user expiration.

35-37. (canceled)

38. The method of claim 1, further comprising updating each of the plurality of parameters to its recommended value for use of the respiratory therapy system during the second period of time.

39-49. (canceled)

50. The method of claim 1, wherein determining the initial value of each of the plurality of parameters includes inputting the user data into a first model and receiving the initial value of each of the plurality of parameters from the first model, wherein the first model is a multi-stage model including:
a first propensity model trained to determine, based on the user data, a propensity score for each of a plurality of distinct combinations of initial values of the plurality of parameters, the propensity score for each respective combination of the plurality of combinations of initial values of the plurality of parameters indicating a probability that the user would be prescribed the respective combination;
a first outcome model trained to determine, based on the user data, a conditional probability that each respective combination of the plurality of combinations of initial values of the plurality of parameters will result in the user satisfying a threshold for a use metric if the user were prescribed the respective combination; and
a first final model trained to determine, based on the propensity score and the conditional probability for each respective one of the plurality of distinct combinations of initial values of the plurality of parameters, a difference between (i) a probability that the respective combination would result in the user satisfying the threshold for the use metric, and (ii) a probability that a default combination of initial values of the plurality of parameters would result in the user satisfying the threshold for the use metric.

51. The method of claim 50, wherein determining the recommended value of each of the plurality of parameters includes inputting the user data and the usage data into a second model and receiving the recommended value of each of the plurality of parameters from the second model, wherein the second model is a multi-stage model including:
a second propensity model trained to determine, based on the user data and the usage data, a propensity score for each of a plurality of distinct combinations of recommended values of the plurality of parameters, the propensity score for each respective combination indicating a probability that the user would be prescribed the respective combination;
a second outcome model trained to determine, based on the user data and the usage data, a conditional probability that each respective one of the plurality of distinct combinations of recommended values of the plurality of parameters will result in the user satisfying a threshold for a use metric if the user were prescribed the respective combination; and
a second final model trained to determine, based on the propensity score and the conditional probability for each respective one of the plurality of distinct combinations of recommended values of the plurality of parameters, a difference between (i) a probability that the respective combination would result in the user satisfying the threshold for the use metric, and (ii) a probability that a default combination of recommended values of the plurality of parameters would result in the user satisfying the threshold for the use metric.

52. (canceled)

53. A system for optimizing a plurality of parameters of a respiratory therapy system, the system comprising a control system configured to implement the method of claim 1.

54-55. (canceled)

56. A system comprising:
a respiratory therapy system configured to supply pressurized air to an individual;
a memory device having stored thereon machine-readable instructions; and
a control system including one or more processors configured to execute the machine-readable instructions to:
receive data associated with a user of the respiratory therapy system;
based at least in part on the user data, determine an initial value of each of a plurality of parameters of the respiratory therapy system, each of the plurality of parameters being associated with a comfort level of the user;
receive usage data associated with use of the respiratory therapy system during one or more sleep sessions in a first period of time in which each of the plurality of parameters has its initial value; and
based at least in part on the user data and the usage data. generate a recommended value of each of the plurality of parameters for use of the respiratory therapy system during a second period of time after the first period of time.

57-107. (canceled)
